(19) United States
(12) Patent Application Publication
(10) Pub. No.: US 2019/0262343 A1
(43) Pub. Date: Aug. 29, 2019

(54) WOUND HEALING USING BRAF INHIBITORS

(71) Applicant: THE REGENTS OF THE UNIVERSITY OF CALIFORNIA, OAKLAND, CA (US)

(72) Inventors: Antoni RIBAS, Los Angeles, CA (US); Helena ESCUIN-ORDINAS, Los Angeles, CA (US)

(21) Appl. No.: 16/312,893

(22) PCT Filed: Jun. 21, 2017
(86) PCT No.: PCT/US2017/038615

§ 371 Date: Dec. 21, 2018

Related U.S. Application Data
(60) Provisional application No. 62/352,976, filed on Jun. 21, 2016.

(51) Int. Cl. A61K 31/517; A61P 17/02; A61K 31/4375; A61K 31/506; A61K 31/519; A61K 31/4409; A61K 31/454; A61K 31/4184; A61K 9/00; A61K 9/06

(52) U.S. Cl. CPC A61K 31/517; A61P 17/02; A61K 31/4375; A61K 31/506; A61K 9/06; A61K 31/4409; A61K 31/454; A61K 31/4184; A61K 9/0014; A61K 31/519

(57) ABSTRACT

Methods for treating a wound are provided herein. Such methods include a step of contacting the wound with an effective amount of a BRAF inhibitor. In some aspects, BRAF inhibitors may be part of a pharmaceutical composition. In such case, the pharmaceutical composition may include an effective amount of a BRAF inhibitor and a pharmaceutically acceptable carrier. In certain aspects, the pharmaceutical composition is a topical agent comprising an ointment, cream liquid, gel, hydrogel, or a spray. Further, in some embodiments, a BRAF inhibitor or a pharmaceutical composition thereof may be part of wound dressing for use in treating a wound. In this case, the wound dressing may be impregnated or coated with the BRAF inhibitor or pharmaceutical composition thereof.

### PRIORITY CLAIM

This application claims priority to U.S. Provisional Application No. 62/352,976, filed Jun. 21, 2016, of which IS incorporated by reference herein, including drawings.

### STATEMENT OF GOVERNMENT INTEREST

This invention was made with Government support under P01 CA168585 and R35 CA197633, each awarded by the National Institute of Health (NIH). The Government has certain rights in the invention.

### BACKGROUND

The serine/threonine-protein kinase B-Raf (“B-Raf” or “BRAF”) is a signal transduction protein kinase that is involved in regulating the MAP kinase/ERKs signaling pathway, affecting cell differentiation, division, and secretion. BRAF^{V600E} is a common oncogenic BRAF mutation, which induces constitutive signaling through the mitogen-activated protein kinase (MAPK) pathway, stimulating cancer-cell proliferation and survival. Clinical development of inhibitors of oncogenic BRAF that block the active conformation of the BRAF kinase, has led to a high rate of objective tumor responses and improvement in overall survival, as compared with standard chemotherapy. Nevertheless, nonmelanoma skin cancers (e.g., well-differentiated cutaneous squamous-cell carcinomas and keratoacanthomas) develop in approximately 15 to 30% of patients treated with BRAF inhibitors such as vemurafenib and dabrafenib (GSK-2118436).

Antitumor activity of BRAF inhibitors such as vemurafenib against BRAF^{V600E}-mutant cells in cell cultures, animal models, and humans is associated with inhibition of oncogenic MAPK signaling, as evidenced by the inhibition of phosphorylated ERK (pERK), a downstream effector of BRAF that is active when phosphorylated. However, BRAF inhibitors induce the opposite effect—that is, increasing pERK in cell lines with wild-type BRAF that harbor upstream pathway activation such as oncogenic RAS or up-regulated receptor tyrosine kinases. This RAF inhibitor-dependent activation of MAPK signaling in BRAF wild-type cells is known as “paradoxical MARK-pathway activation” and is driven by the formation of RAF dimers that lead to signaling through CRAF and consequently MARK-pathway hyperactivation. It would be desirable to harness these skin proliferative side effects of BRAF inhibitors in a non-cancerous setting to accelerate skin wound healing by inducing paradoxical MAPK activation.

### SUMMARY

According to the embodiments described herein, methods for treating a wound caused by a disorder or condition in a subject are provided. Such methods include a step of contacting the wound with an effective amount of a BRAF inhibitor to stimulate wound healing in the subject suffering from the disorder or condition. In certain aspects, the disorder or condition which caused the wound is epidermolysis bullosa (EB), Stevens-Johnson Syndrome (SJS), Toxic Epidermal Necrolysis (TEN), staphylococcal scaled skin syndrome (SSSS), Pemphigus vulgaris (PV), or toxic shock syndrome (TSS).

The BRAF inhibitor may be any suitable agent which inhibits the activity of BRAF including, among other agents, AMG542, ARQ197, ARQ736, AZ628, CEP-32496, GDC-0879, GSK1120212, GSK2118436 (dabrafenib, Tafinlar0), LGX818 (encorafenib), NMS-P186, NMS-P349, NMS-P383, NMS-P396, NMS-P730, PLX3603 (R05212054), PLX4032 (vemurafenib, Zelboraf®), PLX4720 (Difluorophenyl-sulfonamine), PF-04880594, PLX4734, RAF265 (CHIR-265), R04987655, SB590885, sorafenib, sorafenib tosylate, and XL281 (BMS-908662).

In some aspects, BRAF inhibitors may be part of a pharmaceutical composition. In such case, the pharmaceutical composition may include an effective amount of a BRAF inhibitor and a pharmaceutically acceptable carrier. In certain aspects, the pharmaceutical composition is a topical agent comprising an ointment, cream liquid, gel, hydrogel, or a spray. The pharmaceutical composition may also include a second therapeutic agent such as a second pro-angiogenic agent. In some embodiments, the second pro-angiogenic agents may include one or more of (e.g., one of): fibroblast growth factor (FGF, including any FGF member such as FGF-1), vascular endothelial growth factor (VEGF), platelet-derived growth factor (PDGF), placental growth factor (PIGF), an angiopoietin (e.g., Ang1, Ang2), a matrix metalloproteinase (MMP), adelta-like ligand 4 (Dll114), a class 3 Semaphorin (SEMA3), Serpine 1, PECAM1, MMP3, and/or THBS1.

Further, in some embodiments, a BRAF inhibitor or a pharmaceutical composition thereof may be part of wound dressing for use in treating a wound. In this case, the wound dressing may be impregnated or coated with the BRAF inhibitor or pharmaceutical composition thereof. Suitable wound dressings that may be used in accordance with the embodiments described herein include an alginate dressing, an antimicrobial dressing, a bandage, a Band-Aid®, a biosynthetic dressing, a biological dressing, a collagen dressing, a composite dressing, a compression dressing, a contact layer dressing, a foam dressing, a gauze dressing, a hydrocolloid dressing, a hydrogel dressing, a skin sealant or liquid skin dressing, a specialty absorptive dressing, a transparent film dressing, or a wound filler.

### BRIEF DESCRIPTION OF THE DRAWINGS

FIG. 1 is schematic representation illustrating the differential effects of BRAF inhibition in BRAF^{V600E} mutant melanoma (FIG. 1A), BRAF inhibition in BRAF wild type cells in melanoma patients that develop HRAS mutant-derived cutaneous squamous-cell carcinomas and keratoacanthomas (cuSCC/KAs) (FIG. 1B), and BRAF inhibition in BRAF and RAS wild type cells in healthy subjects (FIG. 10).

FIG. 2 illustrates that BRAF inhibition induces paradoxical MAPK activation in human keratinocytes leading to increased proliferation. FIG. 2A is a quantitative analysis of proliferation and scratch healing as the percentage relative wound density of cells at different time points in replicate cultures of HEKa in the presence or absence of vemurafenib by automated microscope analyzer. P value <0.0044 by t-test. Representative images are shown in FIG. 3A. FIG. 2B shows representative images of cell proliferation wound-healing assays of human epithelial adult keratinocytes (HEKa) in the presence or absence of vemurafenib at 0 hours (baseline) and 24 hours. FIG. 2C shows representative images of HEKa cell migration and wound-healing assays in vitro in the presence or absence of mitomycin C (“M”) and/or NSC295642 (“N”) at 0 and 24 hours. FIG. 2D illustrates fold-change representation of colony quantification of HEKa and M249 cells grown in soft-agar with or without exposure to vemurafenib. Representative images are shown in FIG. 3B. FIG. 2E shows the increase in mean spot size for HEKa colonies with or without exposure to vemurafenib. FIG. 2F shows the average number of HEKa colonies with and without vemurafenib and/or trametinib. FIG. 2G is a western blot analyses of pERK and the expression levels of Ki67 in HEKa compared to the BRAF^{V600E} mutant melanoma cell line M249 treated with vemurafenib. FIG. 2H is a western blot analysis showing the levels of pERK and pMEK in HEKa cells compared to the BRAF^{V600E} mutant melanoma cell line M249 when treated with vemurafenib (VEM), trametinib (TRAME), or a combination of VEM and TRAME for 24 hours. FIG. 2I is a phosphoflow cytometry analysis of HEKa and M249 cells treated with vehicle or VEM (1.5 μM) and stained with pERK and Ki67. FIG. 2J shows the quantification of fold-change of pERK and ki67 levels in three replicate cultures of HEKa and M249 cells treated with vemurafenib compared to vehicle. Error bars, mean±s.d.; n=3.

FIG. 3 shows representative results of the experiments described in FIG. 2. FIG. 3A shows time-course images of cell proliferation scratch assays of human epithelial adult keratinocytes (HEKa) in the presence or absence of vemurafenib. Quantitative analysis of proliferation is represented in FIG. 2A. FIG. 3B shows 3D culture images of M249 and HEKa treated with DMSO or VEM. FIG. 3D shows representative photomicrograph hematoxylin and eosin (“H&E”) stained imaged in the presence and absence (Control) of vemurafenib (“VEM”), trametinib (“TRAME”) or a combination of vemurafenib and trametinib (“VEM+TRAME”).

FIG. 4 shows representative phosphoflow cytometry images showing the gating strategy to generate the data presented in FIG. 2I. FIG. 4A shows phosphoflow cytometry images for M249 cells; FIG. 4B shows phosphoflow cytometry images for HEKa cells.

FIG. 5 illustrates that BRAF inhibition accelerates wound healing in mice. FIG. 5A is a schematic representation of the wound-healing assay performed in CH3 mice according to some embodiments. FIG. 5B shows representative images of mice treated topically with vehicle, vemurafenib (VEM, 2 mg), tramatenib (TRAME, 0.2 mg) and the combination of vemurafenib and trametinib (VEM+TRAME) on days 2, 6 and 14. FIG. 5C shows a set of graphs illustrating wound tensile strength (WTS) in three replicate experiments (Vehicle (DMSO/Saline) and VEM; Experiments #1-3), each with 8 mice per group and in a separate experiment comparing vehicle, vemurafenib (VEM), and the combination of VEM and trametinib (TRAME) (Experiment #4). WTS is represented as gram force (gf) per 2 mm strip (p<0.0001 by t-test for all experiments). Error bars mean±s.d.; n=8.

FIG. 6 is a schematic representation of the pathological analysis of wound healing on days 1 (D1), 2 (D2) and 6 (D6) post-treatment. FIG. 6A shows representative photomicrograph H&E images (200×) in the presence and absence of vemurafenib (VEM), trametinib (TRAME) or combination (VEM+TRAME). In each group, the healing of incised wounds involved the same standard processes. Wound-adjacent epidermis undergoes hyperplasia and proliferation and epidermal cells from this process migrate centrally to seal the incised epidermal deficiency. The space of the incision fills initially with fibrin, which is then colonized by fibroblasts, macrophages, polymorphonuclear cells and new capillaries. In the presence of vemurafenib (panels V1, V2, V6) the healing process is accelerated. Wound-adjacent epidermal hyperplasia is more extensive at 2 days post-incision in the vemurafenib group (panel V2) compared to the control specimen (panel C2). Skin surface integrity re-established in 6 days with beginning of sub-epidermal fibrosis (panel V6), while at this point the re-epithelialization is not complete and dermal reparative fibrosis is absent in the control group (panel C6). The group treated with trametinib alone shows slight peri-lesional hyperplasia at day 2 (panel M2) and no evidence of repair by day 6 (panel M6). In the vemurafenib plus trametinib combination group (panels VM1, VM2, VM6) peri-lesional hyperplasia is lower (panel VM2) than in the group treated with vemurafenib at day 2 (panel V2), but greater than trametinib alone (panel M2). Furthermore, re-epithelialization is absent at day 6 (panel VM6). FIG. 6B shows the quantification of the length of epidermal hyperplasia from the right and left side of the wound on days 1, 2 and 6 after treatment with vehicle, vemurafenib, trametinib or combination. Each bar includes data from 4 samples.

FIG. 7 shows gene expression profiling of healing cutaneous wounds in mice with or without exposure to vemurafenib. The top panel shows a heatmap of BRAF signature genes and its overall enrichment score computed using Gene Set Variation Analysis (GVSA); the bottom panel shows a heatmap of wound healing signature genes and the overall GVSA score.

FIGS. 8A-8B show colony number and mean spot size (mm²) quantifications of HEKa grown in soft agar with or without (Control) exposure to vemurafenib (“VEM”), trametinib (“TRAME”) or a combination of vemurafenib and trametinib (“VEM+TRAME”).

FIG. 9A shows representative images of vehicle-treated (“Vehicle”) and vemurafenib-treated (“VEM”) mice on Day 0 and Day 14 following inducement of 6-mm round wounds on the back of Balb/c mice fitted with splinting rings on top of the induced wounds to prevent wound closure by skin contraction. FIG. 9B shows percentage of wound closure on Days 2, 6, and 14 for these mice. FIG. 9C shows representative photomicrograph hematoxylin and eosin (“H&E”), pERK and Ki67 stained images in the presence (“VEM”) and absence (“Vehicle”) of vemurafenib by Day 14. FIG. 9D shows quantification of pERK+ and Ki67+ cells in the vehicle- and vemurafenib-treated wounds on Day 14.

FIG. 10A shows representative photomicrograph immunohistochemistry images on Day 2 (top panel) and Day 6 (bottom panel) of control (“Control”) and vemurafenib-treated (“VEM”) wounds. FIG. 10B shows quantification of pERK+ and Ki67+ cells on the vehicle- and vemurafenib-treated wounds on Day 6 (error bars refer to mean+/−S.D.; p=0.02, n=4).

FIGS. 11A-11B show gene expression profiling of cutaneous wounds in mice with (“VEM”) or without (“CTRL”) exposure to vemurafenib compared to an early wound healing signature (FIG. 11A) and to a postoperative signature (FIG. 11B).

FIG. 12 shows inhibition of BRAF by administration of vemurafenib (“VEM”) activates multiple cell subsets involved in wound healing in the skin compared to control (“CTRL”).

FIG. 13 shows that macrophages increase in mice wounds treated with vemurafenib (“VEM”) and are reversed in the presence of trametinib (“TRAME”). FIG. 13A shows representative photomicrograph immunohistochemistry images of CD68+ cells in an excisional wound splinting model of mice treated with vehicle (“Vehicle”) or vemurafenib (“VEM”) on Day 6 post-treatment; arrows indicate wound areas. FIG. 13B shows quantification of CD68+ cells on Day 6 (p=0.14 by t-test; n=4). FIG. 13C shows representative photomicrograph immunohistochemistry images of CD68+ cells in an excisional wound splinting model of mice treated with vehicle (“Vehicle”), vemurafenib (“VEM”), trametinib (“TRAME”), or a combination of vemurafenib and trametinib (“VEM+TRAME”); arrows indicate wound areas. FIG. 13D shows quantification of CD68+ cells on Day 6 (p=0.0018 by one-way anova; n=4). Error bars in FIGS. 13B and 13D indicate mean+/−SD.

FIG. 14A shows significantly activated biological process/pathways by day 6 of vemurafenib treatment based on the enriched gene ontology (GO) clusters visualized by ClueGO. FIG. 14B shows an integrated view highlighting specific wound healing cell subsets (red; with signature enrichments), their upregulated genes (yellow) and enriched wound healing related processes (blue) in the transcriptome of mice wounds treated with vemurafenib by day 6. Gene node size represents induction at day 6, with the largest node representing a log 2 (FC) of 4.54 and smallest node representing a log 2 (FC) of 1.03. FIG. 14C shows a Representative photomicrograph immunohistochemistry images of IL-6+ cells in an incisional wound model and bar graph, on the right side, representing the quantification of IL-6+ cells on the vehicle- and vemurafenib-treated wounds on day 6 (p=0.02 by t-test n=4). FIG. 14D shows representative photomicrograph immunohistochemistry images of COX-2+ cells and bar graph, on the right side, representing the quantification of COX-2+ cells on the vehicle- and vemurafenib-treated wounds on day 6 (p=0.01 by t-test; n=4). Error bars, mean±s.d. FIG. 14E shows mRNA levels of Egr-1, TNFAIP3 and F7 of total skin wounds treated and untreated with vemurafenib at day 6 post-incision. Bar graphs represent experimental mean of three biological replicates and error bars represent standard error of the mean (s.e.m.). RNA levels were normalized against δ-actin. *** p=0.0006 (Egr-1) and 0.0002 (F7) and ** p=0.0023 (TNFAIP3), all by t-test.

FIG. 15A shows representative photomicrograph immunohistochemistry images of PE-CAM-1+ cells in a excisional wound splinting model of mice treated with vehicle or vemurafenib on day 6 post-treatment and bar graph, on the right side, representing the quantification of PECAM-1+ cells on the vehicle- and vemurafenib-treated wounds on day 6 (p=0.006 by t-test; n=4). Arrows indicate wound areas. FIG. 15B shows representative photomicrograph immunohistochemistry images of PE-CAM-1+ cells in an incisional wound model of mice treated with vehicle, vemurafenib (VEM), trametinib (TRAME) or the combination (VEM+TRAME); (p=0.0002 by one-way anova). Trametinib alone completely depleted the number of PECAM-1+ cells. Double head arrows indicate wound areas. Error bars in FIGS. 15A-15B correspond to mean±SD.

FIG. 16A shows Representative images of mice from each study group on week 15 of treatment. Topical application of 7,12-Dimethylbenz[a]anthracene (DMBA) to FvB/N mice followed by 12-O-tetradecanoylphorbol-13-acetate (TPA) induced skin papillomas and squamous cell carcinomas by week 8 of treatment. In the control (DMBA+TPA) group all eight mice developed tumors. In the groups with DMBA or acetone control, followed by topical application of vemurafenib (VEM, 2 or 4 mg per mice) no skin tumors were induced. FIG. 16B represents tumor count and percentage of tumor incidence per week in graphical form. Error bars in FIG. 16B refer to mean±SD; n=8.

FIG. 17 is a table including a list of genes involved in the post-operative and early wound healing signatures.

### DETAILED DESCRIPTION

Methods, pharmaceutical compositions, and wound dressings for treating wounds using a BRAF inhibitor are provided herein. According to the embodiments described herein, BRAF inhibitors may be used in alone, as part of a pharmaceutical composition; or as part of a wound dressing to accelerate wound healing.

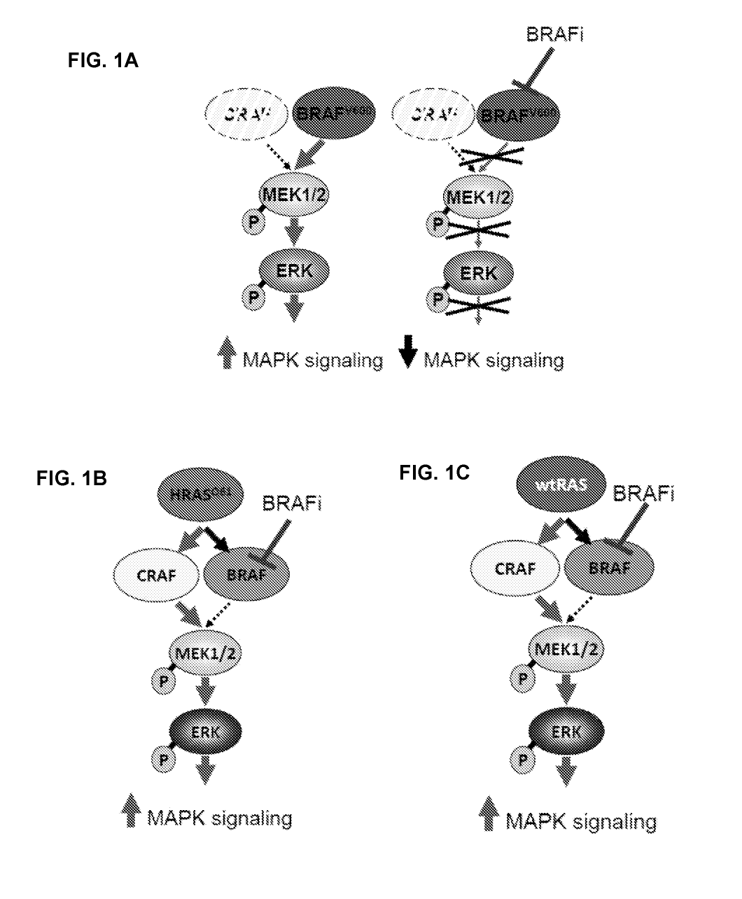

Currently, BRAF inhibitors are used to exploit their anti-proliferative activity in relation to mutated forms of BRAF in diseases and conditions such as cancer (FIG. 1A). However, it has been observed that patients treated with BRAF inhibitors for cancers such as melanoma develop secondary proliferative conditions in spite of the BRAF inhibitor's anti-proliferative effect on mutated forms of BRAF.

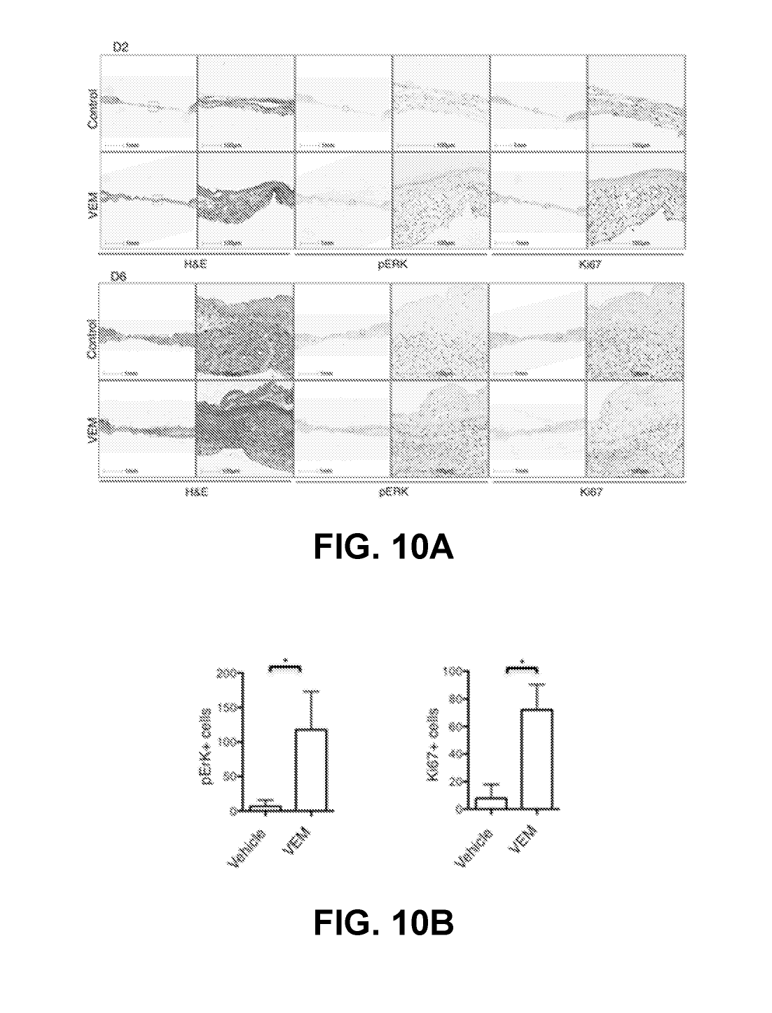

Paradoxical MAPK activation is the pathogenic basis behind the development of these secondary proliferative conditions (e.g., invasive squamous cell carcinomas and keratoacanthomas) in patients treated with BRAF inhibitors (Su et al. 2012; Oberholzer et al. 2012). The frequent presence of RAS mutations upstream of non-mutated BRAF in these secondary skin lesions results in strong RAS-GTP activation, which leads to a paradoxically increased phosphorylation of ERK, increased MAPK pathway output and enhanced cell proliferation (FIG. 1B). Paradoxical MAPK activation is a property of RAF inhibitors (Hall-Jackson et al. 1999) where preferential binding to a BRAF protomer results in transactivation of its CRAF heterodimer partner in the setting of strong upstream RAS-GTP signaling (Heidorn et al. 2010; Poulikakos et al. 2010; Holderfield et al. 2013). As a result, patients with BRAF mutant metastatic melanoma on BRAF inhibitor therapy develop a variety of other skin proliferative conditions (Belum et al. 2013), most of which improve when administering a MEK inhibitor concomitantly (Flaherty et al. 2012), which blocks the downstream effect of paradoxical RAF activation (Su et al. 2012; Escuin-Ordinas et al. 2013). In the Examples below, it is demonstrated that this mechanistic understanding of the skin proliferative side effects of BRAF inhibitors in cancer treatment can be exploited in otherwise healthy subjects (i.e., wild type (wt) RAS and BRAF) to accelerate skin wound healing by inducing paradoxical MAPK activation in wild type cells (FIG. 10).

### BRAF Inhibitors

BRAF inhibitors that may be used in accordance with the embodiments described herein may include any agent which selectively inhibits at least a portion of the biological activity (e.g., signal transduction activity) of a wild type BRAF or a mutant form of BRAF (e.g., BRAF^{V600E}, BRAF^{V600K}, BRAF^{V600D}, BRAF^{V600L}, BRAF^{V600R}). In some aspects, the BRAF inhibitors may be selective for BRAF alone, or may have inhibitory activity against one or more additional targets in the RAF/MEK/ERK pathway. For example in one aspect, the BRAF inhibitor may be a RAF kinase inhibitor, i.e., the inhibitor may have inhibitory activity against RAF kinases such as ARAF, CRAF, or both, in addition to BRAF. In certain embodiments, the BRAF inhibitor is selected to have increased paradoxical MAPK activation activity. As such, the BRAF inhibitors used in accordance with the embodiments described herein may act as a MAPK paradox activator, meaning that the BRAF inhibitor causes an increase in MAPK signaling. In some aspects, a MAPK paradox activator is a BRAF inhibitor that exhibits increased MAPK signaling when the target BRAF kinase is a wild type BRAF kinase. Further, as described in the working Examples below, the beneficial effects by the BRAF inhibitors used in accordance with the embodiments described herein are at least in part caused by improvement of angiogenesis during the proliferative stage of wound healing.

Several BRAF kinase inhibitors have been described in the art, any of which may be suitable for use in the methods, dressings and compositions described herein. Suitable BRAF inhibitors may include, but are not limited to, 1,2-di-cyclyl substituted alkyne compounds or derivatives; 1-methyl-5-(2-(5-(trifluoromethyl)-1H-imidazol-2-yl)pyridin-4-yloxy)-N-(4-(trifluoromethyl)phenyl)-1H-benzo[d]imidazol-2-amine); 2,6-disubstituted quinazoline, quinoxaline, quinoline, and isoquinoline compounds or derivatives; 4-amino-5-oxo-8-phenyl-5H-pyrido-[2,3-D]-pyrimidine compounds or derivatives; 4-amino-thieno[3,2-C]pyridine-7-carboxylic acid compounds or derivatives; 5-(4-aminophenyl)-isoquinoline compounds or derivatives; benzene sulfonamide thiazole compounds or derivatives; benzimidazole compounds or derivatives; bicyclic compounds or derivatives; bridged, bicyclic heterocyclic or spiro bicyclic heterocyclic derivatives of pyrazolo[1,5-a]pyrimidine compounds or derivatives; cinnamide and hydro-cinnamide compounds or derivatives; di-substituted imidazole compounds or derivatives; fused tricyclic pyrazolo[1,5-a]pyrimidine compounds or derivatives; heteroaryl compounds or derivatives; heterocyclic compounds or derivatives; 1H-benzo [D] imidazole compounds or derivatives; imidazo [4,5-B] pyridine compounds or derivatives; N-(6-aminopytidin-3-yl)-3-(sulfonamido) benzamide compounds or derivatives; N-[3-(1-amino-5,6,7,8-tetrahydro-2,4,4B-triazafluoren-9-yl)-phenyl] benzamide compounds or derivatives; nitrogen-containing bicyclic heteroaryl compounds or derivatives; N-oxides of heterocyclic substituted bisarylurea compounds or derivatives; omega-carboxylaryl substituted diphenyl urea compounds or derivatives; oxazole compounds or derivatives; phenethylamide compounds or derivatives; phenylsulfonamide-substituted, pyrazolo[1,5-a]pyrimidine compounds or derivatives; phenyltriazole compounds or derivatives; heterocyclic compounds or derivatives; 1h-pyrazolo[3,4-b] pyridine compounds or derivatives; purine compounds or derivatives; pyrazole [3,4-B] pyridine compounds or derivatives; pyrazole compounds or derivatives; pyrazoline compounds or derivatives; pyrazolo [3,4-b] pyridines, pyrrolo [2,3-b] pyridine compounds or derivatives; pyrazolo [3,4-d]pyrimidine compounds or derivatives; pyrazolo [5,1-c] [1,2,4] triazine compounds or derivatives; pyrazolyl compounds or derivatives; pyrimidine compounds or derivatives; pyrrol compounds or derivatives; pyrrolo [2,3-B] pyridine compounds or derivatives; substituted 6-phenyl-pyrido [2,3-D] pyrimidin-7-ones compounds or derivatives; substituted benzazole compounds or derivatives; substituted benzimidazole compounds or derivatives; substituted bisaryl-urea compounds or derivatives; thienopyridine compounds or derivatives; thienopyrimidine, thienopyridine, or pyrrolopyrimidine compounds or derivatives; thiophene amide compounds or derivatives, and any other suitable aryl and/or heteroaryl compounds or derivatives. In some aspects, the suitable BRAF inhibitors described herein may include the compound or derivative itself or may be a pharmaceutically acceptable salt or solvate thereof.

Several patents and patent applications disclose exemplar BRAF inhibitors that may be used in accordance with the embodiments described herein including, but not limited to, International Patent Application Publication Nos WO2011117381, WO2011119894, WO2011117381, WO2011097594, WO2011097526, WO2011085269, WO2011090738, WO2011025968, WO2011025927, WO2011023773, WO2011028540, WO2010111527, WO2010104973, WO2010100127, WO2010078408, WO2010065893, WO2010032986, WO2009115572, WO2009108838, WO2009111277, WO2009111278, WO2009111279, WO2009111280, WO2009108827, WO2009111260, WO2009100536, WO2009059272, WO2009039387, WO2009021869, WO2009006404, WO2009006389, WO2008140850, WO2008079277, WO2008055842, WO2008034008, WO2008115263, WO2008030448, WO2008028141, WO2007123892, WO2007115670, WO2007090141, WO2007076092, WO2007067444, WO2007056625, WO2007031428, WO2007027855, WO2007002433, WO2007002325, WO2006125101, WO2006124874, WO2006124780, WO2006102079, WO2006108482, WO2006105844, WO2006084015, WO2006076706, WO2006050800, WO2006040569, WO2005112932, WO2005075425, WO2005049603, WO2005037285, WO2005037273, WO2005032548; and U.S. Pat. Nos. 8,642,759, 8,557,830, 8,504,758, 7,863,288, 7,491,829, 7,482,367, and 7,235,576, the specifications of all of which are hereby incorporated by reference as if fully set forth herein.

In certain embodiments, the BRAF inhibitor may be selected from a group of molecules selected from AMG542, ARQ197, ARQ736, AZ628, CEP-32496, GDC-0879, GSK1120212, GSK2118436 (dabrafenib, Tafinlar0), LGX818 (encorafenib), NMS-P186, NMS-P349, NMS-P383, NMS-P396, NMS-P730, PLX3603 (R05212054), PLX4032 (vemurafenib, Zelboraf®), PLX4720 (Difluorophenyl-sulfonamine), PF-04880594, PLX4734, RAF265 (CHIR-265), R04987655, SB590885, sorafenib, sorafenib tosylate, or XL281 (BMS-908662).

In some embodiments, the BRAF inhibitor has a structure of Formula (I) or Formula (II):

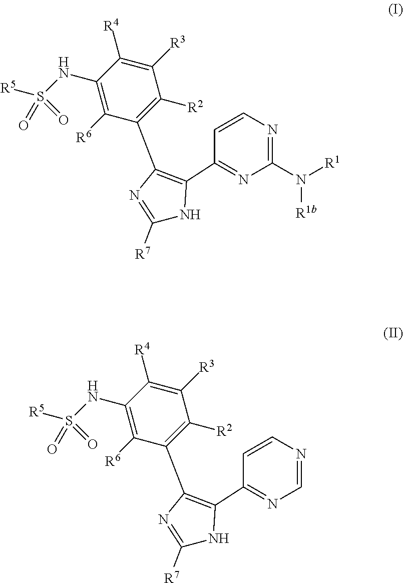

wherein:

R¹ is H, C3-C6 cycloalkyl optionally substituted with cyano, C1-C3 alkyl optionally substituted with cyano, —C(O)NH₂, hydroxy, —X¹NHC(O)OR^{1a}, —X¹NHC(O)NHR^{1a}, where X¹ is C1-C4 alkylene optionally substituted with 1 to 3 groups each independently selected from halo, C1-C4 alkyl or halosubstituted C1-C4 alkyl and R^{1a} is H, C1-C4 alkyl, or halosubstituted C1-C4 alkyl;

R^{1b} is H or methyl;

R² is H or halogen;

R³ is H, halogen, C1-C4 alkoxy, C1-C4 alkyl, halosubstituted C1-C4 alkoxy, or halosubstituted C1-C4 alkyl;

R⁴ is halogen, H, or C1-C4 alkyl;

R⁵ is C1-C6 alkyl, C3-C6 cycloalkyl, C3-C8 branched alkyl, halosubstituted C1-C6 alkyl, halosubstituted C3-C8 branched alkyl, C3-C6 cycloalkyl-(C1-C3)-alkylene, or phenyl, where said phenyl is optionally substituted with 1 to 3 substituents each independently selected form halo, CH₃, or CF₃,

R⁶ is H, C1-C4 alkyl, or halogen; and

R⁷ is H, C1-C6 alkyl, C3-C6 cycloalkyl, 1-methyl-(C3-C6)-cycloalkyl, 1-(halosubstituted-methyl)-(C3-C6)-cycloalkyl, C3-C8 branched alkyl, halosubstituted C1-C6 alkyl, halosubstituted C3-C8 branched alkyl, or phenyl, where said phenyl is optionally substituted with 1 to 3 substituents selected form halogen, C1-C4 alkyl or halosubstituted C1-C4 alkyl, preferably wherein R⁷ is H, C1-C6 alkyl, C3-C6 cycloalkyl, 1-methyl-(C3-C6)-cycloalkyl, C3-C8 branched alkyl, or phenyl, where said phenyl is optionally substituted with 1 to 3 substituents selected form halogen, C1-C4 alkyl or halosubstituted C1-C4 alkyl; or a pharmaceutically acceptable salt thereof.

In one particular embodiment of a compound of Formula (I), R¹ is C1-C3 alkyl optionally substituted with cyano, —C(O)NH₂, hydroxy, —X¹NHC(O)OR^{1a}, where X¹ is C1-C4 alkylene optionally substituted with 1 to 3 groups each independently selected from halo, C1-C4 alkyl, or halosubstituted C1-C4 alkyl and R^{1a} is H, C1-C4 alkyl, or halosubstituted C1-C4 alkyl;

R² is H or halogen;

R³ is H, halogen, C1-C4 alkoxy, C1-C4 alkyl, halosubstituted C1-C4 alkoxy or halosubstituted C1-C4 alkyl;

R⁴ is halogen, H, or C1-C4 alkyl;

R⁵ is C1-C6 alkyl, C3-C6 cycloalkyl, C3-C8 branched alkyl, halosubstituted C1-C6 alkyl, or halosubstituted C3-C8 branched alkyl;

R⁶ is H, C1-C4 alkyl, or halogen; and

R⁷ is H, C1-C6 alkyl, C3-C6 cycloalkyl, 1-methyl-(C3-C6)-cycloalkyl, 1-(halosubstituted-methyl)-(C3-C6)-cycloalkyl, C3-C8 branched alkyl, halosubstituted C1-C6 alkyl, or halosubstituted C3-C8 branched alkyl or phenyl, where said phenyl is optionally substituted with 1 to 3 substituents selected form halogen, C1-C4 alkyl or halosubstituted C1-C4 alkyl, preferably wherein R⁷ is H, C1-C6 alkyl, C3-C6 cycloalkyl, 1-methyl-(C3-C6 cycloalkyl, or phenyl, wherein said phenyl is optionally substituted with 1 to 3 substituents selected form halogen, C1-C4 alkyl or halosubstituted C1-C4 alkyl; or a pharmaceutically acceptable salt thereof.

In a preferred embodiment, a compound of Formula (II) is provided wherein

R¹ is —CH₂—(S)—CH(CH₃)NHC(O)OCH₃,

R^{1b} is H;

R² is H;

R³ is Cl;

R⁴ is H;

R⁵ is CH₃,

R⁶ is F; and

R⁷ is isopropyl, or a pharmaceutically acceptable salt thereof (also referred to herein as “LGX818” or “encorafenib”).

In another embodiment, compounds of Formula (II) are provided wherein

R² is H or F;

R³ is H, halogen, C1-C2 alkoxy, C1-C2 alkyl, halosubstituted C1-C2 alkoxy, or halosubstituted C1-C2 alkyl;

R⁴ is H or methyl;

R⁵ is C1-C4 alkyl, C3-C6 cycloalkyl, C3-C5 branched alkyl, halosubstituted C1-C4 alkyl, halosubstituted C3-C6 branched alkyl, or C3-C6 cycloalkyl-(C1-C3)-alkylene;

R⁶ is H, C1-C2 alkyl, or halogen; and

R⁷ is C3-C6 cycloalkyl, 1-methyl-(C3-C6)-cycloalkyl, or C3-C6 branched alkyl; or a pharmaceutically acceptable salt thereof.

In another embodiment, compounds of Formula (II) are provided wherein

R² is H;

R³ is H, Cl, F, methoxy, methyl, or difluoromethoxy;

R⁴ is H;

R⁵ is methyl, cyclopropyl, ethyl, propyl, isopropyl, sec-butyl, isobutyl, trifluoromethyl, or 3,3,3-trifluoropropyl;

R⁶ is H, methyl, F, or Cl; and

R⁷ is t-butyl, cyclopropyl, or 1-methylcyclopropyl; or a pharmaceutically acceptable salt thereof.

In some embodiments, the BRAF inhibitor is a compound of Formula (III):

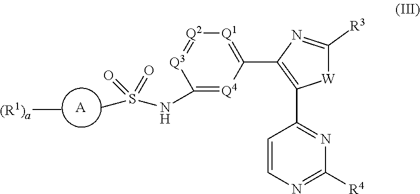

wherein:

a is 0, 1, 2 or 3;

each R¹ is the same or different and is independently selected from halo, alkyl, haloalkyl, —OR⁶, —CO₂R⁶, —NR⁶R⁷, and —ON;

Ring A is selected from C3-C6 cycloalkyl, phenyl, 5-6 membered heterocycle and 5-6 membered heteroaryl, said heterocycle and said heteroaryl each having 1 or 2 heteroatoms selected from N, O and S;

each of Q¹, Q², Q³ and Q⁴ is CH, CR² or N, wherein not more than one of Q¹, Q², Q³ and Q⁴ is N;

each R² is the same or different and is independently selected from halo, alkyl, haloalkyl, and —OR⁶,

W is selected from —O— and —S—;

R³ is selected from H, alkyl, haloalkyl-, -alkylene-OH, —NR⁵R⁷, —C3-C6 cycloalkyl, -alkylene-C(O)—OH, -alkylene-NH₂, and Het;

wherein when R³ is C3-C6 cycloalkyl, said C3-C6 cycloalkyl is optionally substituted with 1 or 2 substituents which are the same or different and are independently selected from halo, C1-C3 alkyl, halo-(C1-C3)-alkyl, OH, O—(C1-C3)-alkyl, oxo, S—(C1-C3)-alkyl), SO₂, NH₂, N(H)(C1-C3)-alkyl and N(C1-C3alkyl)₂,

Het is a 5-6 membered heterocycle having 1 or 2 heteroatoms selected from N, O and S and optionally substituted with 1 or 2 substituents which are the same or different and are each independently selected from halo, C1-C3 alkyl, halo-(C1-C3)-alkyl, O—(C1-C3)-alkyl, C1-C3 alkylene-O—(C1-C3)-alkyl, OH, C1-C3 alkylene-OH, oxo, SO₂((C1-C3)-alkyl), C1-C3 alkylene-SO₂((C1-C3)-alkyl), NH₂, N(H)((C1-C3)-alkyl), N(C1-C3 alkyl)₂, CN, and —CH₂CN,

R⁴ is selected from H, alkyl, haloalkyl, alkenyl, —OR⁶, —R⁵—OR⁶, —R⁵—CO2R⁶, —R⁵—SO₂R⁶, —R⁵—Het, —R⁵—C(O)-Het, —N(H)R⁸, —N(CH3)R⁸, and —R⁵—NR⁶R⁷; each R⁵ is the same or different and is independently C1-C4 alkylene;

each R⁶ and each R⁷ is the same or different and is independently selected from H, alkyl, haloalkyl, —C(O)-alkyl, and —C(O)-cycloalkyl;

R⁸ is selected from H, alkyl (optionally substituted by —OH), haloalkyl, C3-C6 cycloalkyl, —R⁵—(C3-C6)-cycloalkyl, Het², —R⁵—Het², —R⁵—OR⁶, —R⁵—O—R⁵—OR⁶, —R⁵—C(O)₂R⁶, —R⁵—C(O)NR⁶R⁷, —R⁵—N(H)C(O)—R⁶, —R⁵—N(H)C(O)—R⁵—OR⁶, —R⁵—N(H)C(O)₂—R⁵—R⁵—NR⁵R⁷, —R⁵—S(O)₂R⁶, —R⁵—CN, and —R⁵—N(H)S(O)₂R⁶;

wherein when R⁸ is C3-C6 cycloalkyl, said C3-C6 cycloalkyl is optionally substituted with 1 or 2 substituents which are the same or different and are independently selected from halo, C1-C3 alkyl, halo-(C1-C3)-alkyl, OH, O—(C1-C3)-alkyl, oxo, S—(C1-C3)-alkyl, SO₂(C1-C3 alkyl), NH₂, N(H)—(C1-C3)-alkyl and N(C1-C3 alkyl)₂, and N(H)SO₂—(C1-C3)-alkyl, and

Het² is a 4-6 membered heterocycle having 1 or 2 heteroatoms selected from N, O and S and optionally substituted with 1, 2, 3, 4 or 5 C1-C3 alkyl or 1 or 2 substituents which are the same or different and are each independently selected from halo, C1-C3 alkyl, halo-(C1-C3)-alkyl, O—(C1-C3)-alkyl, C1-C3 alkylene-O—(C1-C3 alkyl), OH, C1-C3 alkylene-OH, oxo, SO₂(C1-C3 alkyl), C1-C3 alkylene-SO₂(C1-C3 alkyl), NH₂, N(H)—(C1-C3 alkyl), N(C1-C3 alkyl)₂, N(H)SO₂—(C1-C3 alkyl), C(O)(C1-C3 alkyl), CO₂(C1-C4 alkyl), CN, and —CH₂CN;

and R⁹ and R¹⁹ are independently selected from H and alkyl, and pharmaceutically acceptable salts thereof.

In a preferred embodiment, a compound of Formula (III) is provided wherein

a is 2;

R¹ is F;

each R² is F;

R³ is t-butyl;

R⁴ is N(H)R⁸;

R⁸ is H; and

W is S (referred to herein as “GSK2118436,” “dabrafenib,” or “Tafinlar0”), or a pharmaceutically acceptable salt thereof.

In some embodiments, the BRAF inhibitor is a compound of Formula (IV):

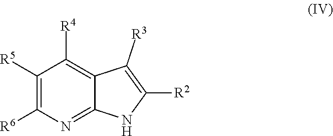

wherein:

R², R⁴, R⁵, and R⁶ are independently selected from the group consisting of hydrogen, halogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted heteroaryl, —CN, —NO₂, —CR^{a}R^{b}R²⁶, and -LR²⁶;

R³ is selected from the group consisting of hydrogen, halogen, optionally substituted lower alkyl, optionally substituted lower alkenyl, optionally substituted lower alkynyl, optionally substituted cycloalkyl, optionally substituted heterocycloalkyl, optionally substituted aryl, optionally substituted heteroaryl, —CN, —NO₂, —CR^{a}R^{b}R²⁶, -LR²⁶ and -A-Ar-L1-R²⁴;

A is selected from the group consisting of —O—, -5-, —CR^{a}R^{b}—, —NR¹—, —C(O)—, —C(S)—, —S(O)—, and —S(O)₂—,

R¹ is selected from the group consisting of hydrogen, lower alkyl, cycloalkyl, heterocycloalkyl, aryl, heteroaryl, —C(O)R⁷, —C(S)R⁷, —S(O)₂R⁷, —C(O)NHR⁷, —C(S)NHR⁷, and —S(O)₂NHR⁷, wherein lower alkyl is optionally substituted with one or more substituents selected from the group consisting of fluoro, —OH, —NH₂, lower alkoxy, lower alkylthio, mono-alkylamino, di-alkylamino, and —NR⁸R⁹, wherein the alkyl chain(s) of lower alkoxy, lower alkylthio, mono-alkylamino, or di-alkylamino are optionally substituted with one or more substituents selected from the group consisting of fluoro, —OH, —NH₂, lower alkoxy, fluoro substituted lower alkoxy, lower alkylthio, fluoro substituted lower alkylthio, mono-alkylamino, di-alkylamino, and cycloalkylamino, provided, however, that any substitution of the alkyl chain carbon bound to 0 of alkoxy, S of thioalkyl or N of mono- or di-alkylamino is fluoro, further provided, however, that when R¹ is lower alkyl, any substitution on the lower alkyl carbon bound to the N of —NR¹— is fluoro, and wherein cycloalkyl, heterocycloalkyl, aryl or heteroaryl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, fluoro substituted lower alkylthio, mono-alkylamino, di-alkylamino, and cycloalkylamino; R⁷ is selected from the group consisting of lower alkyl, cycloalkyl, heterocycloalkyl, aryl, and heteroaryl, wherein lower alkyl is optionally substituted with one or more substituents selected from the group consisting of fluoro, —OH, —NH₂, lower alkoxy, lower alklylthio, mono-alkylamino, di-alkylamino, and —KR⁸R⁹, provided, however, that any substitution of the alkyl carbon bound to the N of —C(O)NHR⁷, —C(S)NHR⁷ or —S(O)₂NHR⁷ is fluoro, wherein the alkyl chain(s) of lower alkoxy, lower alkylthio, mono-alkylamino, or di-alkylamino are optionally substituted with one or more substituents selected from the group consisting of fluoro, —OH, —NH₂, lower alkoxy, fluoro substituted lower alkoxy, lower alkylthio, fluoro substituted lower alkylthio, mono-alkylamino, di-alkylamino, and cycloalkylamino, provided, however, that any substitution of the alkyl chain carbon bound to O of alkoxy, S of thioalkyl or N of mono- or di-alkylamino is fluoro, and wherein cycloalkyl, heterocycloalkyl, aryl and heteroaryl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, fluoro substituted lower alkylthio, mono-alkylamino, di-alkylamino, and cycloalkylamino;

Ar is selected from the group consisting of optionally substituted arylene and optionally substituted heteroarylene;

L at each occurrence is independently selected from the group consisting of -(alk)ₐ-S-(alk)_{b}-, -(alk)ₐ-O-(alk)_{b}-, -(alk)ₐ-NR²⁵-(alk)_{b}-, -(alk)ₐ˜C(O)-(alk)_{b}-, -(alk)ₐ-C(S)-(alk)_{b}-, -(aUc)ₐ-S(O)-(alk)_{b}-, -(alk)ₐ-S(O)₂-(alk)_{b}-, -(alk)ₐ-OC(O)-(alk)_{b}-, -(alk)ₐ-C(O)O-(alk)_{b}-, -(alk)ₐ-OC(S)-(alk)_{b}-, -(alk)ₐ-C(S)O-(alk)_{b}-, -(alk)ₐ-C(O)NR²⁵-(alk)_{b}-, -(alk)ₐ-C(S)NR²⁵-(alk)_{b}-, -(alk)ₐ-S(O)₂NR²⁵-(alk)_{b}-, -(alk)ₐ-NR²⁵C(O)-(alk)_{b}-, -(alk)ₐ-NR²⁵C(S)-(alk)_{b}-, -(alk)ₐ-NR²⁵S(O)₂-(alk)_{b}-, -(alk)ₐ-NR²⁵C(O)O-(alk)_{b}-, -(alk)ₐ-NR²⁵C(S)O-(alk)_{b}-, -(alk)ₐ-OC(O)NR²⁵-(alk)_{b}-, -(alk)ₐ-OC(S)NR²⁵-(alk)_{b}-, -(alk)ₐ-NR²⁵C(O)NR²⁵-(alk)_{b}-, -(alk)ₐ-NR²⁵C(S)NR²⁵-(alk)_{b}-, and -(alk)ₐ-NR²⁵S(O)2NR²⁵-(alk)_{b}-, a and b are independently 0 or 1; alk is C1-C3 alkylene or C1-C3 alkylene substituted with one or more substituents selected from the group consisting of fluoro, —OH, —NH₂, lower alkyl, lower alkoxy, lower alkylthio, mono-alkylamino, di-alkylamino, and —NR⁸R⁹, wherein lower alkyl or the alkyl chain(s) of lower alkoxy, lower alkylthio, mono-alkylamino or di-alkylamino are optionally substituted with one or more substituents selected from the group consisting of fluoro, —OH, —NH₂, lower alkoxy, fluoro substituted lower alkoxy, lower alkylthio, fluoro substituted lower alkylthio, mono-alkylamino, di-alkylamino and cycloalkylamino, provided, however, that any substitution of the alkyl chain carbon bound to O of alkoxy, S of thioalkyl or N of mono- or di-alkylamino is fluoro;

L1 is —(CR^{a}R^{b})ᵥ— or L, wherein v is 1, 2, or 3; wherein R^{a} and R^{b} at each occurrence are independently selected from the group consisting of hydrogen, fluoro, —OH, —NH₂, lower alkyl, lower alkoxy, lower alklylthio, mono-alkylamino, di-alkylamino, and —NR⁸R⁹, wherein the alkyl chain(s) of lower alkyl, lower alkoxy, lower alkylthio, mono-alkylamino, or di-alkylamino are optionally substituted with one or more substituents selected from the group consisting of fluoro, —OH, —NH₂, lower alkoxy, fluoro substituted lower alkoxy, lower alkylthio, fluoro substituted lower alkylthio, mono-alkylamino, di-alkylamino, and cycloalkylamino, provided, however, that any substitution of the alkyl chain carbon bound to O of alkoxy, S of thioalkyl or N of mono- or di-alkylamino is fluoro; or any two of R^{a} and R^{b} on the same or different carbons combine to form a 3-7 membered monocyclic cycloalkyl or 5-7 membered monocyclic heterocycloalkyl and any others of R^{a} and R^{b} are independently selected from the group consisting of hydrogen, fluoro, —OH, —NH₂, lower alkyl, lower alkoxy, lower alklylthio, mono-alkylamino, di-alkylamino, and —NR⁸R⁹, wherein the alkyl chain(s) of lower alkyl, lower alkoxy, lower alkylthio, mono-alkylamino, or di-alkylamino are optionally substituted with one or more substituents selected from the group consisting of fluoro, —OH, —NH2, lower alkoxy, fluoro substituted lower alkoxy, lower alkylthio, fluoro substituted lower alkylthio, mono-alkylamino, di-alkylamino, and cycloalkylamino, provided, however, that any substitution of the alkyl chain carbon bound to O of alkoxy, S of thioalkyl or N of mono- or di-alkylamino is fluoro, and wherein the 3-7 membered monocyclic cycloalkyl or 5-7 membered monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, fluoro substituted lower alkylthio, mono-alkylamino, di-alkylamino, and cycloalkylamino;

R⁸ and R⁹ combine with the nitrogen to which they are attached to form a 5-7 membered heterocycloalkyl optionally substituted with one or more substituents selected from the group consisting of fluoro, —OH, —NH₂, lower alkyl, fluoro substituted lower alkyl, lower alkoxy, fluoro substituted lower alkoxy, lower alkylthio, and fluoro substituted lower alkylthio;

R²⁵ at each occurrence is independently selected from the group consisting of hydrogen, optionally substituted lower alkyl, optionally substituted cycloalkyl, optionally substituted heterocycloalkyl, optionally substituted aryl, and optionally substituted heteroaryl; and

R²⁴ and R²⁶ at each occurrence are independently selected from the group consisting of hydrogen, provided, however, that hydrogen is not bound to any of S(O), S(O)₂, C(O) or C(S) of L or Li, optionally substituted lower alkyl, optionally substituted lower alkenyl, provided, however, that when R²⁴ or R²⁶ is optionally substituted lower alkenyl, no alkene carbon thereof is bound to N, S, O, S(O), S(O)₂, C(O) or C(S) of L or L1, optionally substituted lower alkynyl, provided, however, that when R²⁴ or R²⁶ is optionally substituted lower alkynyl, no alkyne carbon thereof is bound to N, S, O, S(O), S(O)₂, C(O) or C(S) of L or L1, optionally substituted cycloalkyl, optionally substituted heterocycloalkyl, optionally substituted aryl, and optionally substituted heteroaryl.

In a preferred embodiment, a compound of Formula (III) is provided wherein:

R² is H;

R³ is -A-Ar-L1-R²⁴;

A is —C(O)—;

Ar is 2,4-difluorophenyl;

L1 is —SO₂—;

R⁴ is H;

R⁵ is 4-chlorophenyl;

R⁶ is H;

R²⁴ is n-propyl (referred to herein as “PLX4032” “vemurafenib,” or “Zelboraf®”) or a pharmaceutically acceptable salt thereof.

In other embodiments, one skilled in the art may generate or identify novel BRAF inhibitors using in vitro, in vivo, in silico, or other screening methods known in the art. For example, a BRAF inhibitor of wild type BRAF may be identified from a training set of small molecules, peptides, or nucleic acids using an assay for detecting phosphorylation of molecules which are downstream from BRAF in the MAPK signaling cascade (e.g., MEK and/or ERK). The BRAF inhibitor may act to suppress or inhibit BRAF expression and/or signaling function, thereby reducing phosphorylation of MEK and ERK. Several phosphorylation assays are available which could be used in such embodiments including, but not limited to, kinase activity assays (e.g., those sold by R&D Systems®, Promega®, Life Technologies®); phospho-specific antibodies for use with immunoassays such as western blots, enzyme-linked immunosorbent assays (ELISA), flow cytometry, immunocytochemistry, immunohistochemistry; mass spectrometry, proteomics, and phospho-protein multiplex assays. In certain embodiments, BRAF inhibitors for use in the embodiments described herein may be identified using screening methods which measure candidate inhibitor ability to activate the MAPK pathway. This activation of the MAPK pathway may be accomplished by transactivating CRAF. In contrast to typical BRAF inhibitor screening for use in treatment of cancer and other diseases associated with aberrant BRAF expression, BRAF inhibitors identified in this manner (also referred to herein as MAPK paradox activators) may be used to take advantage of paradoxical MAPK activation to accelerate cutaneous wound healing by inducing increased proliferation of skin cells.

As used herein, the term “pharmaceutically acceptable salt” means those salts of compounds of the invention that are safe and effective for application in a subject and that possess the desired biological activity. Pharmaceutically acceptable salts include salts of acidic or basic groups present in compounds of the invention. Pharmaceutically acceptable salts include, but are not limited to, hydrofluoride, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,11-methylene-bis-(2-hydroxy-3-naphthoate)), aluminum, calcium, lithium, magnesium, potassium, sodium, zinc, and diethanolamine salts. Certain compounds of the invention can form pharmaceutically acceptable salts with various amino acids. For a review on pharmaceutically acceptable salts see Berge, et al., 66 J. Pharm. Sci. 1-19 (1977), which is incorporated herein by reference.

### Pharmaceutical Compositions

In some embodiments, one or more of the BRAF inhibitors described above may be part of a pharmaceutical composition. In some aspects, the pharmaceutical composition includes at least one BRAF inhibitor and a pharmaceutically acceptable carrier. The term “pharmaceutically acceptable carrier” as used herein means a pharmaceutically-acceptable material, composition or vehicle, such as a liquid or solid filler, diluent, excipient, solvent or encapsulating material, involved in carrying or transporting a BRAF inhibitor from one location, body fluid, tissue, organ (interior or exterior), or portion of the body, to another location, body fluid, tissue, organ, or portion of the body.

In some embodiments, the pharmaceutical composition comprises a pharmaceutically acceptable carrier and a BRAF inhibitor that is consistent with Formula (I) or Formula (II) or a pharmaceutically acceptable salt thereof. In some embodiments, the BRAF inhibitor is LGX818 (encorafenib) or a salt or derivative thereof.

In some embodiments, the pharmaceutical composition comprises a pharmaceutically acceptable carrier and a BRAF inhibitor that is consistent with Formula (III) or a pharmaceutically acceptable salt thereof. In some embodiments, the BRAF inhibitor is GSK2118436 (dabrafenib, Tafinlar0) or a salt or derivative thereof.

In some embodiments, the pharmaceutical composition comprises a pharmaceutically acceptable carrier and a BRAF inhibitor that is consistent with Formula (IV) or a pharmaceutically acceptable salt thereof. In some embodiments, the BRAF inhibitor is PLX4032 (vemurafenib, Zelboraf®) or a salt or derivative thereof.

Each carrier is “pharmaceutically acceptable” in the sense of being compatible with the other ingredients, e.g., a BRAF inhibitor, of the formulation and suitable for use in contact with the tissue or organ of a biological system without excessive toxicity, irritation, allergic response, immunogenicity, or other problems or complications, commensurate with a reasonable benefit/risk ratio.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) alcohol, such as ethyl alcohol and propane alcohol; (20) phosphate buffer solutions; and (21) other non-toxic compatible substances employed in pharmaceutical formulations such as acetone.

The pharmaceutical compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. In addition, the formulation for the pharmaceutical composition may also include wetting agents, coloring agents, release agents, coating agents, perfuming agents, preservatives, antioxidants, or other auxiliary ingredients.

In one embodiment, the pharmaceutically acceptable carrier is an aqueous carrier, e.g. buffered saline and the like. In certain embodiments, the pharmaceutically acceptable carrier is a polar solvent, e.g. acetone and alcohol. In certain aspects, the pharmaceutically acceptable carrier is of a suitable material which allows, facilitates, or enhances transdermal, topical, aerosol, inhalable, or any other suitable mode of administration, such as those routes of administration described in detail below.

The concentration of BRAF inhibitors in these formulations can vary widely, and will be selected primarily based on fluid volumes, viscosities, body weight and the like in accordance with the particular mode of administration selected and the biological system's needs. Generally, the amount of the BRAF inhibitor or inhibitors present in the pharmaceutical composition will be that which will produce a therapeutic effect. For example, in some embodiments, the weight per volume (w/v) or weight percent (wt %) concentration of a BRAF inhibitor or inhibitors in the pharmaceutical composition may be between approximately 0.001% to 100%, 0.001% to 90%, 0.001% to 80%, 0.001% to 70%, 0.001% to 60%, 0.001% to 50%, 0.001% to 40%, 0.001% to 30%, 0.001% to 20%, 0.001% to 10%, 0.001% to 1%, 0.01% to 100%, 0.01% to 90%, 0.01% to 80%, 0.01% to 70%, 0.01% to 60%, 0.01% to 50%, 0.01% to 40%, 0.01% to 30%, 0.01% to 20%, 0.01% to 10%, 0.01% to 1%, 0.1% to 100%, 0.1% to 90%, 0.1% to 80%, 0.1% to 70%, 0.1% to 60%, 0.1% to 50%, 0.1% to 40%, 0.1% to 30%, 0.1% to 20%, 0.1% to 10%, 0.1% to 1%, 1% to 100%, 1% to 90%, 1% to 80%, 1% to 70%, 1% to 60%, 1% to 50%, 1% to 40%, 1% to 30%, 1% to 20%, 1% to 10%, 1% to 5%, 1% to 4%, 1% to 3%, 1% to 2%, 0.1% to 0.9%, 0.1% to 0.8%, 0.1% to 0.7%, 0.1% to 0.6%, 0.1% to 0.5%, 0.1% to 0.4%, 0.1% to 0.3%, 0.1% to 0.2%, 0.2% to 1%, 0.3% to 1%, 0.4% to 1%, 0.5% to 1%, 0.6% to 1%, 0.7% to 1%, 0.8% to 1%, or 0.9% to 1%.

In other embodiments, the concentration of a BRAF inhibitor or inhibitors in the pharmaceutical composition may be approximately 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM. 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 200 mM, 300 mM, 400 mM, 500 mM, 600 mM, 700 mM, 800 mM, 900 mM, or 1M. In some aspects, the concentration (molarity or wt %) of a BRAF inhibitor that produces a therapeutic effect in a subject (e.g., a human or other mammal) can be extrapolated from in vitro or in vivo data, from cell culture and/or animal experiments, such as those described in the Examples below.

In some aspects, the pharmaceutical composition also includes at least one additional therapeutic agent. In addition to one or more BRAF inhibitors, a suitable therapeutic agent may be included as part of the pharmaceutical composition. In certain embodiments, the therapeutic agent is a second pro-angiogenic agent. Suitable second pro-angiogenic agents may include, but is not limited to, fibroblast growth factor (FGF, including all FGF members such as FGF-1), vascular endothelial growth factor (VEGF), platelet-derived growth factor (PDGF), placental growth factor (PIGF), angiopoietins (Ang1, Ang2), matrix metalloproteinases (MMPs), delta-like ligand 4 (Dll4), and class 3 Semaphorins (SEMA3s), Serpine 1, PECAM1, MMP3, and/or THBS.

Other suitable therapeutic agents that may be included as part of the pharmaceutical composition include, but are not limited to, wound treatment agents such as growth factors (e.g., recombinant platelet derived growth factor (PDGF; Regranex®/Becaplermin gel)), fish skin-based MariGen Omega3 tissue-regeneration technology, sugar, antacids, vitamin A, vitamin D, antimicrobials and antiseptics (e.g., acetic acid, acidified nitrite, acticoat 7, aquacel-Ag, antimicrobial peptides, bacitracin, BCTP nanoemulsion, cadexomer iocide, iodine, centrimide, chlorhexidine, essential oils, flammacerium, FPQC, fusidic acid, gentamicin, gluconate, hexachlorophene, honey, iodine compounds, iodine tincture, liposomal iodine, mafenide acetate, metronidazole, mupirocin, mupirocin calcium, neomycin sulfate, neosporin, nitrofurazone, nystatin, phage therapy, papaya, probiotics, polymixin B, povidone iodine, retapamulin, sodium hypochlorite, hydrogen peroxide, silver, silvercel, silver amniotic membrane, silver nitrate, silver dressings, silver foams, silver sulfadiazine, sulfacetamide Na⁺, and superoxidized water); and analgesics such as rubefacients (e.g., salicylate, nicotinate, capsaicin, capsicum extracts), NSAIDs (e.g., ibuprofen, diclofenac, felbinac, ketoprofen, piroxicam, naproxen, flubiprofen), hydrocortisone, benzalkonium chloride, benzydamine, mucopolysaccharide polysulphate, salicylamide, phenol, cooling sprays, calamine, and local anesthetics (e.g., lidocaine, lignocaine, prilocaine, benzocaine, pramoxine, dibucaine).

### Wound Dressings

The BRAF inhibitors and pharmaceutical compositions thereof which are described herein may be used in combination with or in conjunction with one or more wound dressings. In certain embodiments, one or more BRAF inhibitors or a pharmaceutical composition thereof is used to impregnate or coat a wound dressing. Any wound dressing, such as those described below, may be impregnated or coated with one or more BRAF inhibitors or a pharmaceutical composition that includes one or more BRAF inhibitors. Such pharmaceutical compositions are described in detail above.

In one embodiment, wound dressings that are impregnated or coated with a pharmaceutical composition that includes one or more BRAF inhibitors may be sold as a single wound-healing dressing or a set of wound-healing dressings that are individually wrapped. In such case, the dressing and BRAF inhibitor(s) are supplied together in a single dressing unit which, when applied to a wound, serves not only confer typical wound-healing properties of the dressing (e.g., stops bleeding, reduces pain, protects from further harm or injury, protects from infection), but also acts to enhance and/or accelerate wound healing functions.

Several suitable wound dressings are known and used in the art to promote wound healing, protect open wounds, provide pain relief, and to prevent infection and/or contamination, any of which may be used in accordance with the embodiments described herein. Examples of suitable wound dressings include, but are not limited to, alginates, antimicrobials, bandages, Band-Aids®, biosynthetics, biologicals, collagens, composites, compression bandages, contact layers, foams, gauze, hydrocolloids, hydrogels, skin sealants/liquid skin, specialty absorptives, transparent films, wound fillers. In some aspects, more than one wound dressing that is impregnated or coated with one or more BRAF inhibitor may be used on a wound. In other aspects a wound dressing may be used in combination with a topical ointment, gel, spray, paste, liquid or other formulation, each of which may include one or more BRAF inhibitors or compositions thereof.

According to some embodiments, a wound dressing is impregnated or coated with one or more of the BRAF inhibitors described above, alone or as part of a pharmaceutical composition. In certain aspects the one or more BRAF inhibitors that may be used to impregnate or coat a wound dressing are selected from one or more of AMG542, ARQ197, ARQ736, AZ628, CEP-32496, GDC-0879, GSK1120212, GSK2118436 (dabrafenib, Tafinlar0), LGX818 (encorafenib), NMS-P186, NMS-P349, NMS-P383, NMS-P396, NMS-P730, PLX3603 (R05212054), PLX4032 (vemurafenib, Zelboraf®), PLX4720 (Difluorophenyl-sulfonamine), PF-04880594, PLX4734, RAF265 (CHIR-265), R04987655, SB590885, sorafenib, sorafenib tosylate, and XL281 (BMS-908662). The impregnated or coated wound dressing may be applied directly to a wound such that the dressing imparts the therapeutic effect of the one or more BRAF inhibitors to the wound.

In some embodiments, a wound dressing is impregnated or coated with a BRAF inhibitor that is consistent with Formula (I) or Formula (II) or a pharmaceutically acceptable salt thereof, alone or as part of a pharmaceutical composition. In some embodiments, the BRAF inhibitor is LGX818 (encorafenib) or a salt or derivative thereof.

In some embodiments, a wound dressing is impregnated or coated with a BRAF inhibitor that is consistent with Formula (III) or a pharmaceutically acceptable salt thereof, alone or as part of a pharmaceutical composition. In some embodiments, the BRAF inhibitor is GSK2118436 (dabrafenib, Tafinlar0) or a salt or derivative thereof.

In some embodiments, a wound dressing is impregnated or coated with a BRAF inhibitor that is consistent with Formula (IV) or a pharmaceutically acceptable salt thereof, alone or as part of a pharmaceutical composition. In some embodiments, the BRAF inhibitor is PLX4032 (vemurafenib, Zelboraf®) or a salt or derivative thereof.

### Methods of Use

In some embodiments, the BRAF inhibitors described above, alone or as part of a pharmaceutical composition, may be used in methods for treating a wound on a subject. Such methods described herein may be used to treat any type of wound, including, but not limited to, acute non-penetrating wounds (e.g., abrasions, lacerations, contusions), acute penetrating wounds (e.g., stab wounds, superficial cuts, scratches or lacerations, surgical incisions and wounds, gunshot wounds), thermal wounds (e.g., burns, sunburns, and frostbite), ulcers (e.g., chronic diabetic ulcers, pressure ulcers/bedsores), blisters, rashes, chemical wounds, animal or insect bites and stings, and electrical wounds.

In certain embodiments, the methods described herein may be used to treat a wound resulting from or caused by an underlying disorder or condition in the subject. Thus, the BRAF inhibitors described above, alone or as part of a pharmaceutical composition, may be used in methods for treating the disorder or condition. Disorders or conditions that may cause wounds that are treatable by the BRAF inhibitors described above, alone or as part of a pharmaceutical composition, include, but are not limited to, epidermolysis bullosa (EB), Stevens-Johnson Syndrome (SJS), Toxic Epidermal Necrolysis (TEN), staphylococcal scaled skin syndrome (SSSS), Pemphigus vulgaris (PV), and toxic shock syndrome (TSS).

The methods for treating wounds, including those methods for treating an underlying disorder or condition that causes wounds, may include a step of contacting the wound with an effective amount of one or more BRAF inhibitors to accelerate healing of the wound. Suitable BRAF inhibitors that may be used in accordance with the methods described herein include, but are not limited to, those described above. In certain aspects the one or more BRAF inhibitors may be selected from one or more of AMG542, ARQ197, ARQ736, AZ628, CEP-32496, GDC-0879, GSK1120212, GSK2118436 (dabrafenib, Tafinlar0), LGX818 (encorafenib), NMS-P186, NMS-P349, NMS-P383, NMS-P396, NMS-P730, PLX3603 (R05212054), PLX4032 (vemurafenib, Zelboraf®), PLX4720 (Difluorophenyl-sulfonamine), PF-04880594, PLX4734, RAF265 (CHIR-265), R04987655, SB590885, sorafenib, sorafenib tosylate, and XL281 (BMS-908662).

In some embodiments, the BRAF inhibitor that may be used in accordance with the methods described herein is consistent with Formula (I) or Formula (II) or a pharmaceutically acceptable salt thereof. In some embodiments, the BRAF inhibitor is LGX818 (encorafenib) or a salt or derivative thereof.

In some embodiments, the BRAF inhibitor that may be used in accordance with the methods described herein is consistent with Formula (III) or a pharmaceutically acceptable salt thereof. In some embodiments, the BRAF inhibitor is GSK2118436 (dabrafenib, Tafinlar0) or a salt or derivative thereof.

In some embodiments, the BRAF inhibitor that may be used in accordance with the methods described herein is consistent with Formula (IV) or a pharmaceutically acceptable salt thereof. In some embodiments, the BRAF inhibitor is PLX4032 (vemurafenib, Zelboraf®) or a salt or derivative thereof.

According to the methods described herein, contacting a wound with one or more BRAF inhibitors or a pharmaceutical composition thereof may be accomplished by any suitable route of delivery or administration. To treat a wound, a BRAF inhibitor or a pharmaceutical composition thereof may be delivered or administered by any administration route known in the art including, but not limited to, oral, nasal, topical, aerosol, transmucosal, epidermal, transdermal, dermal, ophthalmic, pulmonary, subcutaneous, and/or inhalation. The pharmaceutical compositions can be administered in a variety of unit dosage forms depending upon the method of administration. For example, unit dosage forms suitable for transdermal administration include impregnated or coated patches, bandages, gauze or any other dressings described herein.

According to some embodiments, a BRAF inhibitor or a pharmaceutical composition thereof can be given to a subject in the form of a formulation or preparation suitable for each administration route. The formulations useful in the methods of the invention may include one or more BRAF inhibitors, one or more pharmaceutically acceptable carriers therefor, and optionally one or more additional therapeutic agents or ingredients. The formulations may conveniently be presented in unit dosage form and may be prepared by any methods well known in the art of pharmacy. The amount of active ingredient which can be combined with a carrier material to produce a single dosage form will vary depending upon the subject being treated and the particular mode of administration. The amount of a BRAF inhibitor which can be combined with a carrier material to produce a pharmaceutically effective dose will generally be that amount of a BRAF inhibitor which produces a therapeutic effect.

In some embodiments, formulations may be suitable for oral administration to use for treatment of mouth wounds or sores. In such embodiments, the formulation may be in solid dosage form (e.g., capsules, cachets, pills, tablets, lozenges (using a flavored basis, usually sucrose and acacia or tragacanth), powders, granules), or in liquid dosage form (e.g., as a solution or a suspension in an aqueous or non-aqueous liquid, as an oil-in-water or water-in-oil liquid emulsion or microemulsion, as an elixir or syrup, as pastilles (using an inert base, such as gelatin and glycerin, or sucrose and acacia) and/or as mouth washes and the like), each containing a predetermined amount of a BRAF inhibitor as an active ingredient.

In solid dosage forms for oral administration (e.g., capsules, tablets, pills, dragees, powders, granules and the like), the BRAF inhibitor may be mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate, (5) solution retarding agents, such as paraffin, (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

In liquid dosage forms, the BRAF inhibitor may be mixed with inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming and preservative agents. Additionally, suspensions may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

In some embodiments, formulations for the topical, transdermal, epidermal, or dermal administration of a BRAF inhibitor composition include powders, sprays, ointments, pastes, creams, lotions, gels, solutions, patches, dressings, and inhalants. The active component may be mixed under sterile conditions with a pharmaceutically acceptable carrier, and with any preservatives, buffers, or propellants which may be required. Such ointments, pastes, creams and gels may contain, in addition to the BRAF inhibitor composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to the BRAF inhibitor composition, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

In certain aspects, the BRAF inhibitor or pharmaceutical compositions thereof may be administered by aerosol. This is accomplished by preparing an aqueous aerosol, liposomal preparation or solid particles or powder containing the BRAF inhibitor. A nonaqueous (e.g., fluorocarbon propellant) suspension could be used. Sonic nebulizers can also be used. An aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids (such as glycine), buffers, salts, sugars or sugar alcohols. Aerosols generally are prepared from isotonic solutions.

Transdermal patches or wound dressings can also be used to deliver BRAF inhibitors or pharmaceutical compositions thereof to a site of wound. Examples of wound dressings that may be used are described in detail above. Such formulations can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel.

In some embodiments, the BRAF inhibitor or pharmaceutical composition thereof that is used in the methods to treat wounds is part of a wound dressing. In some aspects, this means that the BRAF inhibitor or pharmaceutical composition thereof is used to coat or impregnate all or a part of a wound dressing as described above. Wound dressings that may be used in accordance with this embodiment include an alginate dressing, an antimicrobial dressing, a bandage, a Band-Aid®, a biosynthetic dressing, a biological dressing, a collagen dressing, a composite dressing, a compression dressing, a contact layer dressing, a foam dressing, a gauze dressing, a hydrocolloid dressing, a hydrogel dressing, a skin sealant or liquid skin dressing, a specialty absorptive dressing, a transparent film dressing, or a wound filler.

The term “effective amount” as used herein refers to an amount of a BRAF inhibitor that produces a desired effect. For example, a population of cells may be contacted with an effective amount of a BRAF inhibitor to study its effect in vitro (e.g., cell culture) or to produce a desired therapeutic effect ex vivo or in vitro. An effective amount of a BRAF inhibitor may be used to produce a therapeutic effect in a subject, such as treating a target condition, alleviating symptoms associated with the condition, or producing a desired physiological effect. For example, an effective amount of a BRAF inhibitor may be an amount that stimulates wound healing. In such a case, the effective amount of a BRAF inhibitor is a “therapeutically effective amount,” “therapeutically effective concentration” or “therapeutically effective dose.” The precise effective amount or therapeutically effective amount is an amount of the BRAF inhibitor that will yield the most effective results in terms of efficacy of treatment in a given subject or population of cells. This amount will vary depending upon a variety of factors, including but not limited to the characteristics of the BRAF inhibitor (including activity, pharmacokinetics, pharmacodynamics, and bioavailability), the physiological condition of the subject (including age, sex, wound type and status, general physical condition, responsiveness to a given dosage, and type of medication) or cells, the nature of the pharmaceutically acceptable carrier or carriers in the formulation, and the route of administration. Further an effective or therapeutically effective amount may vary depending on whether the BRAF inhibitor is administered alone or in combination with a compound, drug, therapy or other therapeutic method or modality. One skilled in the clinical and pharmacological arts will be able to determine an effective amount or therapeutically effective amount through routine experimentation, namely by monitoring a cell's or subject's response to administration of a BRAF inhibitor and adjusting the dosage accordingly. For additional guidance, see Remington: The Science and Practice of Pharmacy, 21^{st} Edition, Univ. of Sciences in Philadelphia (USIP), Lippincott Williams & Wilkins, Philadelphia, Pa., 2005, which is hereby incorporated by reference as if fully set forth herein.

“Treating” or “treatment” of a wound may refer to the use of any agent or dressing to help heal, protect, repair, or restore the structure and function of an acutely or chronically wounded, injured or diseased tissue; an preventing the condition, slowing the onset or rate of development of the condition, preventing or reducing the risk of developing a condition secondary to the wound, killing antimicrobial infections present at the site of the wound, preventing or delaying the development of pain and other symptoms associated with the wound, reducing or ending pain and other symptoms associated with the wound, generating a complete or partial regression of the wound, or some combination thereof.

In some embodiments, a BRAF inhibitor or a pharmaceutical composition thereof as described above may be administered or delivered in combination with or in conjunction with one or more additional therapeutic agents. The BRAF inhibitor and the therapeutic agent(s) can act additively or synergistically together. “In combination,” “in combination with,” or “in conjunction with,” as used herein, means in the course of treating the same wound in the same subject using two or more agents, dressings, drugs, treatment regimens, treatment modalities or a combination thereof, in any order, and in any number of applications. This includes simultaneous administration, as well as in a temporally spaced order of up to several days apart. The two or more agents, dressings, drugs, treatment regimens, treatment modalities or combination thereof may be part of a single application or administration, or may be applied or administered separately. For example, a BRAF inhibitor may be administered as an ingredient of a pharmaceutical composition or formulation. This composition or formulation may include one or more additional therapeutic agents to be applied as a single topical composition, or alternatively, this composition may be applied to a wound with a second pharmaceutical composition or formulation that contains the one or more additional therapeutic agents. Once the composition or formulation is applied, a wound dressing may be applied over the topical composition(s). In another example, a BRAF inhibitor may be used to impregnate a wound dressing alone or as part of a pharmaceutical composition. The combination treatment may also include more than a single administration of any one or more of the agents, drugs, treatment regimens or treatment modalities. Further, the administration of the two or more agents, dressings, drugs, treatment regimens, treatment modalities or a combination thereof may be by the same or different routes of administration.

In one embodiment, BRAF inhibitors (i.e., pro-angiogenic agents) and pharmaceutical compositions thereof may be administered or delivered in combination with or in conjunction with a second pro-angiogenic agent to enhance the BRAF inhibitor's angiogenic effect. Suitable second pro-angiogenic agents may include, but is not limited to, fibroblast growth factor (FGF, including all FGF members such as FGF-1), vascular endothelial growth factor (VEGF), platelet-derived growth factor (PDGF), placental growth factor (PIGF), angiopoietins (Ang1, Ang2), matrix metalloproteinases (MMPs), delta-like ligand 4 (Dll4), and class 3 Semaphorins (SEMA3s), Serpine 1, PECAM1, MMP3, and/or THBS1.

Other suitable therapeutic agents that may be administered or delivered in combination with or in conjunction with BRAF inhibitors and pharmaceutical compositions thereof may include, but are not limited to, wound treatment agents such as growth factors (e.g., recombinant platelet derived growth factor (PDGF; Regranex®/Becaplermin gel)), fish skin-based MariGen Omega3 tissue-regeneration technology, sugar, antacids, vitamin A, vitamin D, antimicrobials and antiseptics (e.g., acetic acid, acidified nitrite, acticoat 7, aquacel-Ag, antimicrobial peptides, bacitracin, BCTP nanoemulsion, cadexomer iocide, iodine, centrimide, chlorhexidine, essential oils, flammacerium, FPQC, fusidic acid, gentamicin, gluconate, hexachlorophene, honey, iodine compounds, iodine tincture, liposomal iodine, mafenide acetate, metronidazole, mupirocin, mupirocin calcium, neomycin sulfate, neosporin, nitrofurazone, nystatin, phage therapy, papaya, probiotics, polymixin B, povidone iodine, retapamulin, sodium hypochlorite, hydrogen peroxide, silver, silvercel, silver amniotic membrane, silver nitrate, silver dressings, silver foams, silver sulfadiazine, sulfacetamide Na⁺, and superoxidized water); and analgesics such as rubefacients (e.g., salicylate, nicotinate, capsaicin, capsicum extracts), NSAIDs (e.g., ibuprofen, diclofenac, felbinac, ketoprofen, piroxicam, naproxen, flubiprofen), hydrocortisone, benzalkonium chloride, benzydamine, mucopolysaccharide polysulphate, salicylamide, phenol, cooling sprays, calamine, and local anesthetics (e.g., lidocaine, lignocaine, prilocaine, benzocaine, pramoxine, dibucaine).

The following examples are intended to illustrate various embodiments of the invention. As such, the specific embodiments discussed are not to be construed as limitations on the scope of the invention. It will be apparent to one skilled in the art that various equivalents, changes, and modifications may be made without departing from the scope of invention, and it is understood that such equivalent embodiments are to be included herein. For example, although the examples below are directed to experiments conducted with treatment with vemurafenib, one skilled in the art would understand that other BRAF inhibitors could be used in lieu of vemurafenib to produce similar results. Further, all references cited in the disclosure are hereby incorporated by reference in their entirety, as if fully set forth herein.

### EXAMPLES

BRAF inhibitors are highly active for the treatment of patients with BRAF^{V600E} mutant metastatic melanoma, with their main side effect being an array of skin proliferative changes from hyperkeratosis to invasive squamous cell carcinomas. The pathogenic basis of these side effects is mediated by paradoxical activation of the MAPK pathway, where BRAF inhibitors increase MAPK pathway signaling in cells that are wild type for BRAF. This phenomenon was exploited in the studies below to accelerate cutaneous wound healing by inducing increased proliferation of skin cells. The BRAF inhibitor vemurafenib accelerated the proliferation and migration of human keratinocytes in scratch assays, which were mediated by increased ERK phosphorylation and cell cycle progression. In a wound-healing mouse model, topically applied vemurafenib improved the tensile strength of healing wounds through paradoxical MAPK activation, as assessed by gene expression profiling. Thus, topical BRAF inhibitors may have applications in accelerating the healing of skin wounds.

### Example 1: Materials and Methods

The following materials and methods were used in the working examples 2-8 below unless otherwise specified.

Cell Proliferation and Migration Assays.

Human epidermal adult keratinocytes (HEKa) were purchased from Invitrogen (Grand Island, N.Y.; C-005-5C). HEKa cells (25,000/well) were plated on 96-well ImageLock cell migration plates (Essen Bioscience, Ann Arbor, Mich.) and incubated overnight. Then, the cell monolayer was scratched with a 96-pin WoundMaker (Essen Bioscience), and the cells washed with PBS prior to adding cell medium. Cells were maintained in culture with a concentration of 1.5 μM of vemurafenib until complete scratch closure. Cell migration was monitored by a microscope gantry inside a cell incubator, which was connected to a networker external controller hard drive that gathered and processed image data (Incucyte, Essen Bioscience, Ann Arbor, Mich.). This allows an automated and non-invasive method of monitoring live cells in culture. HEKa and M249 cells were plated on Oris™ cell migration plates (Platypus Technologies, Madison, Wis.), treated with vehicle, vemurafenib (1.5 μM), trametinib (1 μM), mitomycin C (10 pg/ml), NSC 295642 (1 μg/ml) or combination, and loaded with CellTracker™ Green CMFDA (5-chloromethylfluorescein diacetate) probe (Life Technologies, Carlsbad, Calif.). Mitomycin C and NSC 295642 were purchase from Sigma Aldrich, Saint Louis, Mo. Cell migration was assessed using a BioSpot Series 5 UV analyzer (Cellular Technology Limited, Cleveland, Ohio).

Colony Forming Assays.

Twenty four-well plates were covered with 300 μl of serum-free RPMI 1640 (Fisher Scientific, Hampton, N.H.) with 0.6% Noble agar (BD Biosciences, San Jose, Calif.), and incubated at 37° C. overnight, until solid. 300 μl of a suspension of HEKa and M249 cells (15,000 cells/ml) in a 1:1 mixture of growth medium with a concentration of 1.5 μM of vemurafenib and growth factor-reduced matrigel (BD Biosciences) was added to each well. After one week, automated colony quantification was performed using a BioSpot Series 5 UV analyzer.

Intracellular Flow Cytometry Analysis.

HEKa and M249 cells treated with a concentration of 1.5 μM of vemurafenib or vehicle control were fixed with formaldehyde to a final concentration of 1.6%, and permeabilized using methanol (90%). Then, they were washed in staining buffer (sterile PBS, 0.5% BSA, 0.01% sodium azide, NaN₃), and stained with Alexa Flour 647 mouse anti-ERK1/2 (pT202/pY204) antibody and PE mouse anti-human Ki67 antibody (BD Pharmingen, San Jose, Calif.) as previously described by Comin-Anduix, et al., PLoS One 5:e12711 (2010). After incubation, cells were washed again and resuspended in 3 ml of staining buffer. A total of 30,000 cellular events were acquired for analysis. Data was analyzed using FlowJo (Tree Star Inc., Ashland, Oreg.). HEKa and M249 cells were routinely tested for mycoplasma and were found to be negative.

Western Blotting.

M249 and HEKa cells were treated in duplicate with a concentration of 1.5 μM of vemurafenib or vehicle control. Western blotting was performed as described previously by Escuin-Ordinas, et al., Mol. Oncol. 8:250-60 (2014). Primary antibodies included p-ERK Thr204/205, ERK, Ki67 and beta-actin (all from Cell Signaling Technology, Danvers, Mass.) Immuno-reactivity was revealed with an ECL-Plus kit, using a Typhoon scanner (both from Amersham Biosciences Co, Piscataway, N.J.).

Incisional Dorsal Wound Model.

C3Hf/Kam (H2-k) female mice were used for wound-healing studies at 8-10 weeks of age. They were bred at UCLA and used under the Animal Research Council (ARC) protocol #2013-066-01 “Wound Healing with BRAF inhibitors”. Full-thickness wounds approximately 2.5 cm long were made in the shaved dorsal skin of anesthetized mice as described (Kim et al. 2013), with ketamine/xylazine as an anesthetic. Clinical grade vemurafenib pills (Zelboraf, Genentech, South San Francisco, Calif.) were grinded and dissolved in dimethylsulfoxide (DMSO; Fisher Scientific) and phosphate buffered saline (PBS; 1:4) to a concentration of 40 μg/μl and 50 μl of the mixture (or DMSO in PBS as vehicle control) was added topically. Wounds were closed with 3-4 clips, which were removed after 2 days. Vemurafenib suspension (2 mg) or vehicle control was re-applied topically to the wound site on days 2, 4, 6, 8, 10 and 12. Two weeks after wounding, a square of skin containing the wound was removed from euthanized mice and cut into seven 2-mm strips of 20 mm in length with a multiblade device, so that each 2-mm-wide strip contained a horizontal wound sample (Gorodetsky et al. 1988). The strips were spread on filter papers soaked in ice-cold PBS in covered petri dishes until WTS measurement, as previously described (Gorodetsky 2008) using an Instron tensiometer (Model 3342; Instron, Norwood, Mass.). The skin strips were stretched at a rate of 1 cm min-1 to breaking point to obtain the peak WTS in gf per 2 mm. The WTS of unwounded skin from 12-weeks old mice is 250 (gf) (Gorodetsky et al. 1988).

Excisional Skin Wound Splinting Model.

Seven to nine week old female Balb/c mice were used for these studies under the ACR protocol #2010-011-13F. Mice were anesthetized with 2-3% isoflurane in an induction chamber and kept under anesthesia during the whole surgery. The back of the mice was shaved, washed with betadine and 70% ethanol and a dose of buprenorphine (2.5 mg/kg) was administered, subcutaneously, prior to the surgery. Two excisional wounds were made in the skin aside the midline of the animal using a 6-mm biopsy punch. 20 μl of vemurafenib (0.1 mg/μl) or DMSO was applied topically on the wounds one minute before suturing of the splinting rings. The splinting rings have an 8-mm transparent window, which was covered with Tegaderm to allow visualization and measurement of the wound size. All animals were observed daily for signs of inflammation and pain for the first 48 hours post-surgery. Vemurafenib or DMSO was repeatedly applied on day 2 and 4. Wounds were photographed at day 0, day 2, day 6 and day 14, based on which the percentages of wound closure were calculated.

Histological Analyses.

Dorsal skin wounds from CH3 mice treated with vemurafenib suspension or DMSO in saline control suspension were excised at day 2, 6 and 14. They were fixed in 10% neutral buffered formalin and embedded in paraffin and stained with hematoxylin and eosin (H&E) using standard methods. Balb/c mice were sacrificed at day 2, 6 and 14 with isoflurane overdose. Two 8-mm round pieces of tissue were collected from each Balbc/c mouse containing the whole wound area and the surrounding tissue and skin, cut precisely in half at the midline of the wound and fixed in 1% paraformaldehyde (PFA) for 16-18 hours at 4° C., dehydrated in 70% EtOH, and then paraffin embedded. Sections were cut at 4 μm, deparaffinized with xylene and descendant ethanol, and then incubated in 3% H₂O₂ for 10 minutes. After a wash in distilled water, the slides were incubated for 25 minutes in citrate buffer pH6 (Invitrogen) at 95° C. using a vegetable steamer. The slides were brought to room temperature, rinsed in PBST (Phosphate Buffered Saline containing 0.05% Tween-20), and then incubated at room temperature with 1:100 anti-mouse Ki-67 antibody (DAKO, Carpinteria, Calif.) for 1 hour and 1:10 phospho-ERK Ab (Cell Signaling), overnight. The Ki67 stained slides were rinsed with PBST and incubated at room temperature with 1:200 polyclonal Rabbit anti-rat immunoglobulin/Biotinylated Ab (Dako, E0468) for 30 minutes. All the slides were rinsed with PBST, and incubated with Dako EnVision+ System-HRP Labelled Polymer Anti-Rabbit (Dako) at room temperature for 30 minutes. After a rinse with PBST, the slides were incubated with DAB (3,3′-Diaminobenzidine) for visualization. Subsequently, the slides were washed in tap water, counterstained with Harris' Hematoxylin, dehydrated in ethanol, and mounted with media. The imaging and quantification of the cell-based immunohistochemistry, was performed with the HALO Next Generation Imaging analysis software (Indica Labs; Corrales, N. Mex.). HALO measures and reports individual cell data maintaining an interactive link between cell metrics and cell imagery. The number of pERK+, Ki67+ and PECAM-1+ cells was automatically counted with the HALO software. Three 20× fields of view from each side of the wound were automatically counted for pERK and Ki67 stains. PECAM-1+ and CD68+ cells were automatically counted on each side of the wound edges where the granulation tissue starts (1 mm length each side) on the excisional wound splinting model, and in the entire wound area on the incisional wound model.

RNAseq Analysis.

RNA from mice skin samples in each treatment group were extracted (RNeasy Mini Kit, Qiagen, Valencia, Calif.) on days 2, 6 and 14 and sent for RNAseq analysis using 2×100 bp paired end Illumina HiSeq2000 (Illumina, San Diego, Calif.) sequencing run. Raw sequences were mapped to the mouse mm9 reference sequence by TOPHAT. The normalized gene expression levels of each were expressed in FPKM values as generated by the program cuffquant and cuffnorm on TOPHAT's BAM output 20. The options “--frag-bias-correct”, “--multi-read-correct” and “--compatible-hits-norm” were applied on the cuffquant run. The heatmap of the MAPK and wound-healing signatures was generated based on the signature genes' row-normalized FPKM levels by the R package gplots. GSVA score was computed using normalized read counts as previously described (Hanzelmann et al. 2013). Normalized read counts (computed by Cuffnorm on the RNAseq BAM files) of the mouse tissue with/without treatment of vemurafenib at day 2, 6 and 14 were supplied to the dermDB database (dermDB database described in Inkeles et al. 2015). Different immune cell type gene sets that were used were reported previously (Jacomy et al. 2014). Enrichment scores were computed as the average difference of each gene in a gene set against its mean across all samples. Row-normalized enrichment scores of the immune gene sets were visualized and the p-values of the enrichment score differences were computed based on a null distribution generated by permutation of the gene labels (n=100000). P-values were adjusted using Benjamini-Hochberg method. Specific biological processes on day 6 with vemurafenib treatment (with control day 6 as reference) were nominated using gene ontology (GO) enrichment analysis on the upregulated genes (min. 2-fold upregulation) in the treated group. The enriched GO terms were computed and visualized using ClueGO (Bindea, G. et al., (2009)). The integrated panel highlighting relations among enriched genes, gene processes and specific immune subsets was created using the Gephi software (Abel et al. 2009). The data was submitted to the GEO repository (accession number GSE74558).

Quantitative Polymerase Chain Reaction.

Q-PCR was performed using a one-step reverse transcription kit developed specifically for SYBR® Green-based real-time PCR (Power SYBR® Green RNA-to-CT™ 1-Step Kit, Thermo Fisher Scientific, Carlsbad, Calif.), with a standard quantitation-comparative Ct procedure as set by the manufacturer. Triplicate reactions (25 μl) of each experimental sample were prepared with the following primers: TNFAIP3 (Fwd: 5′-CTGACCTGGTCCTGAGGAAG-3′; Rev: 5′-GCAAAGTCCTGTTTCCA-3′), F7 (Fwd: 5′ GACTTTGACGGTCGGAACTGTG 3′; Rev: 5′ GCGGCTGCTGGAGTTTCTTT 3′) and Egr-1 (Fwd: 5-GACGAGTTATCCCAGCCAAA-3, Rev: 5-GGCAGAGGAAGACGATGAAG-3). Data were normalized to Bactin levels.

Carcinogenesis Studies.

Female FVB/N mice were purchased from Charles River Laboratory (Wilmington, Mass.). Tumor induction procedures were carried out in accordance with ARC protocol #2013-066. The two-stage carcinogenesis procedures were performed as described previously by Abel, et al., Nature protocols 4:1350-62 (2009); and Ishikawa, et al., Mol. Oncol. 4:347-56 (2010) with 8 mice per group. DMBA and TPA were purchased from Sigma. Clinical grade vemurafenib pills were grinded and dissolved in DMSO; to a concentration of 0.02 and 0.04 mg/μl and 100 μl of the mixtures (or DMSO as vehicle control) was added topically on the back of the mice. Vemurafenib suspension (2 or 4 mg) or vehicle control was re-applied topically to the back of the mice twice a week for 15 weeks.

Statistical Analysis.

Data were analyzed with GraphPad Prism (version 5) software (GraphPad Software, La Jolla, Calif.). Significance was determined by unpaired two-tailed Student's t-test or one-way analysis of variance (ANOVA). Variance was similar between the groups that were statistically compared.

### Example 2: BRAF Inhibitor Enhances Regrowth of Keratinocytes to Cover In Vitro Scratch Site

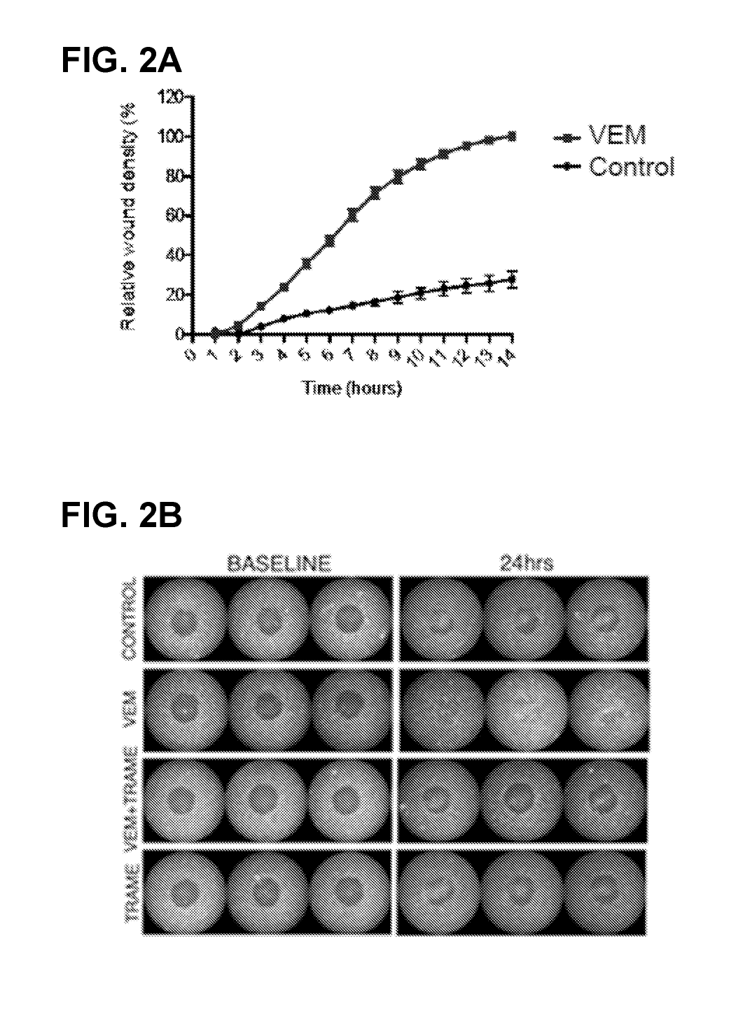

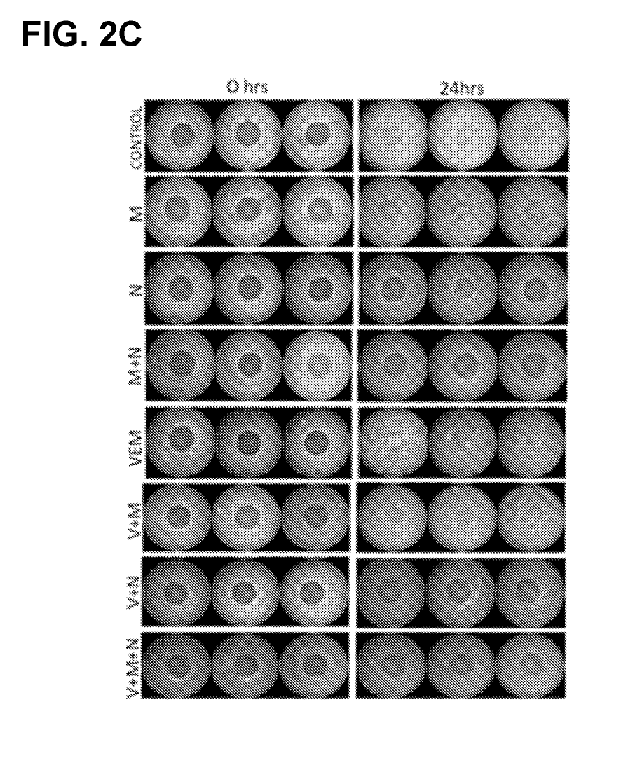

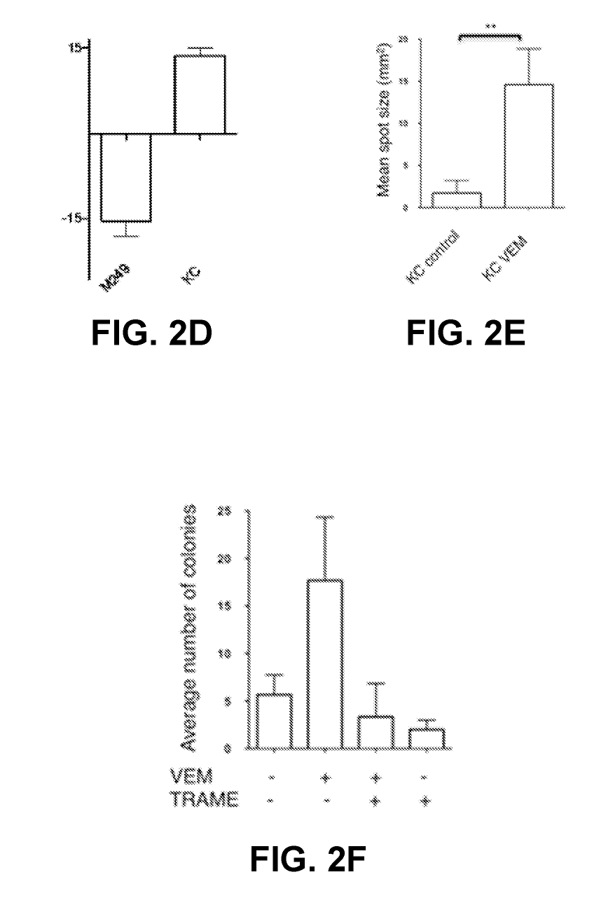

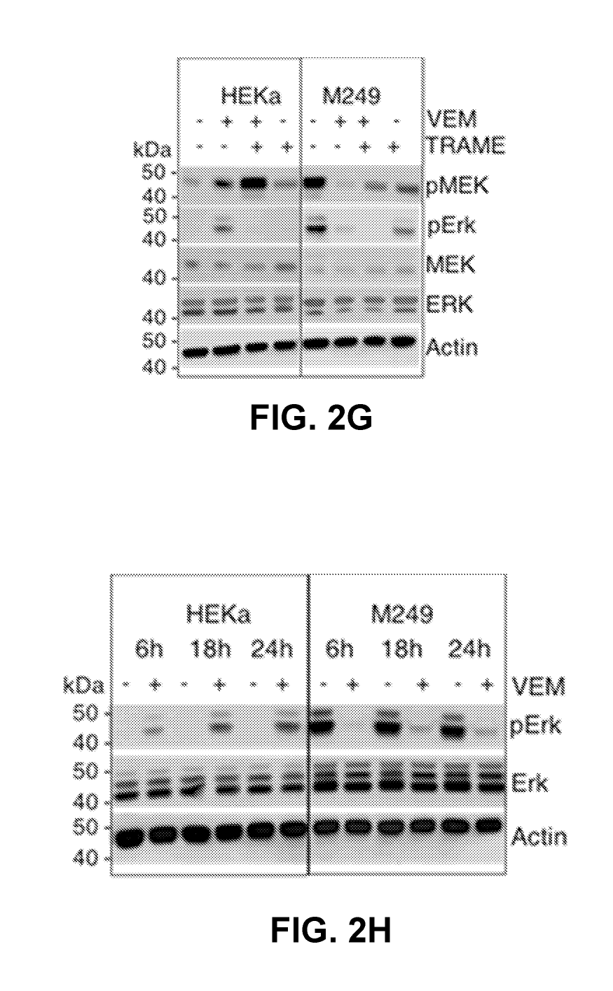

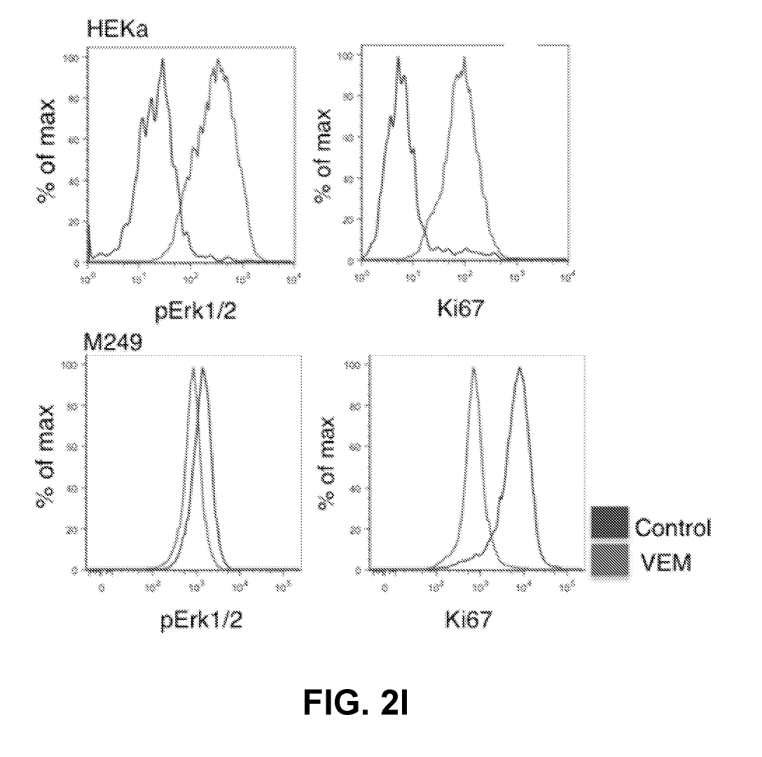

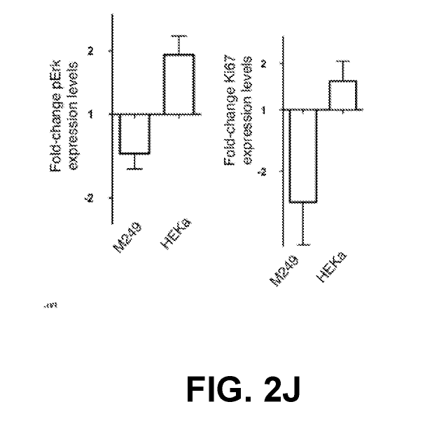

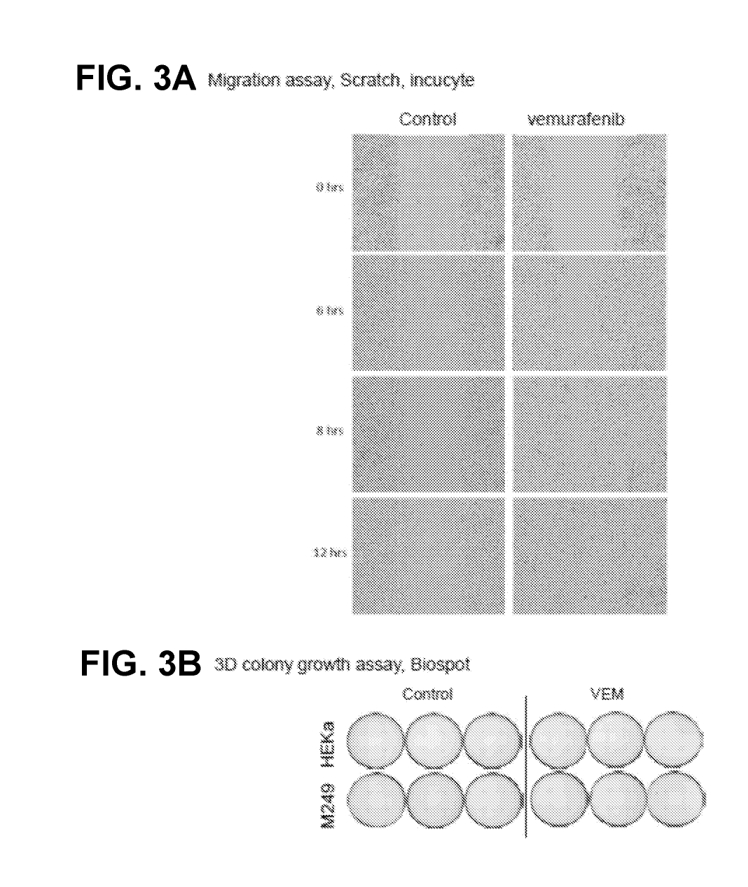

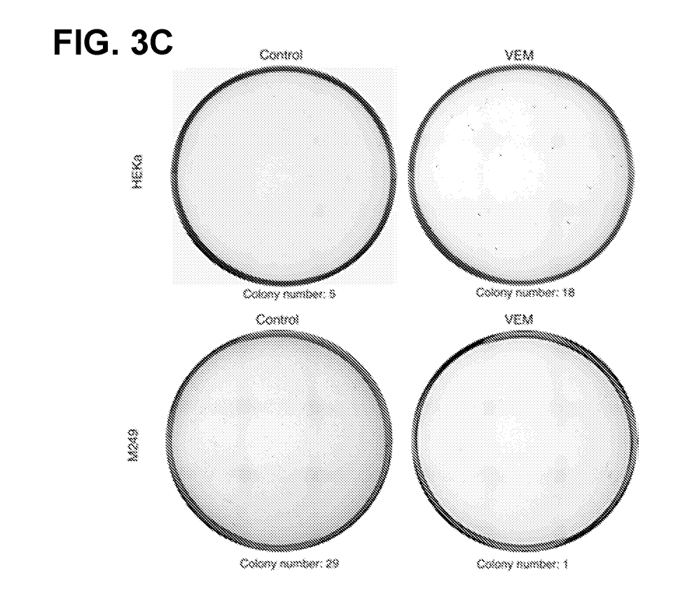

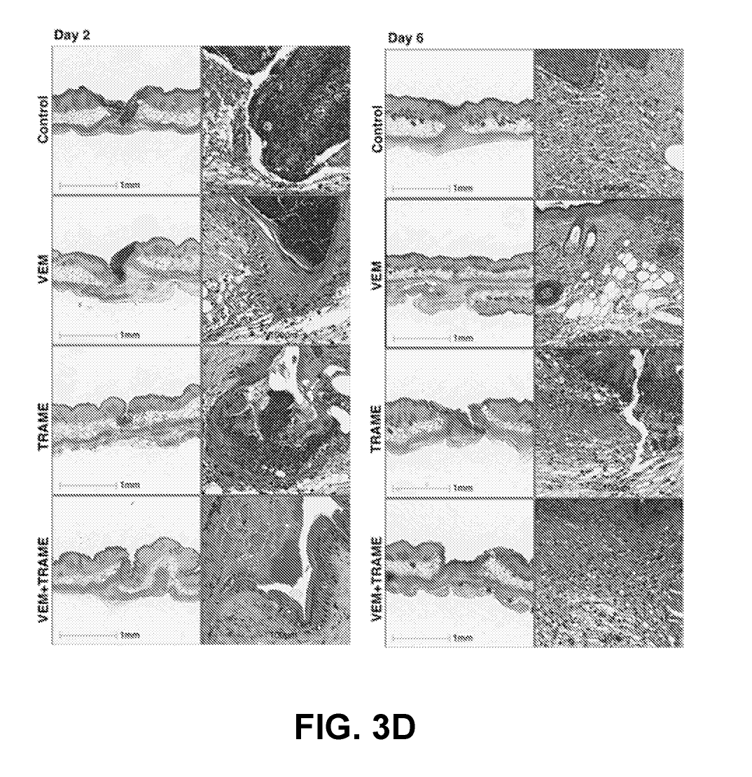

Human epithelial adult keratinocytes (HEKa) cultured as a monolayer in 96-well plates were subject to a scratch assay, where proliferating keratinocytes should regrow and cover the scratch. Replicate cultures with or without the BRAF inhibitor vemurafenib were placed in an incubator with an automated microscope analyzer and the number of nucleated cells in the original scratch was recorded over time. The presence of vemurafenib induced a statistically significant improvement in the covering of the original scratch, which was clearly evident at 6, 8 and 12 hours after start of the study (FIG. 2A and FIG. 3A). The proliferative advantage of HEKa cultured in the presence of vemurafenib was also evident using 96 well plates with seeder stoppers in the middle of each well; proliferating keratinocytes treated with vemurafenib covered the center of the wells after 24 hours, while control treated wells continue to be devoid of cells in the middle (FIG. 2B). The enhanced migration was inhibited by adding trametinib, a MEK inhibitor, to the cultures treated with vemurafenib (FIG. 2B; “TRAME”).

Vemruafenib (1.5 μM) also induced both proliferative and migratory effects on HEKa cells in vitro as combination cultures containing 10 μg/mL of mitomycin C, a mitosis inhibitor (FIG. 2C, “M”), or in combination cultures containing 1 μg/mL of NSC295642, an inhibitor of cell motility (FIG. 2C, “N”) in an assay in which migration and growth were initiated by removal of a central space sealant.

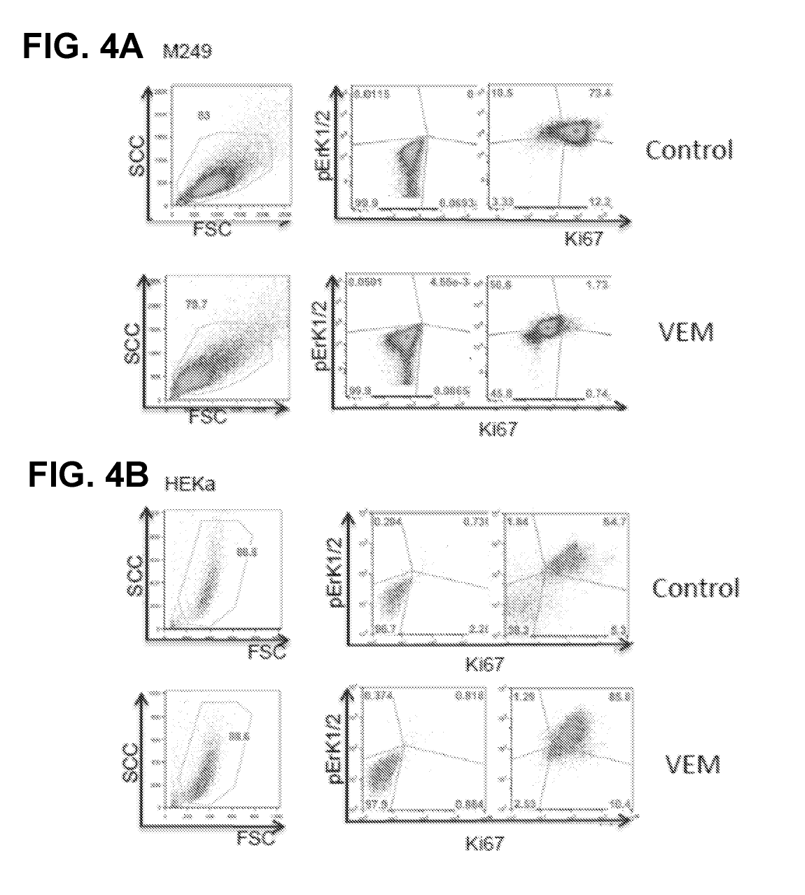

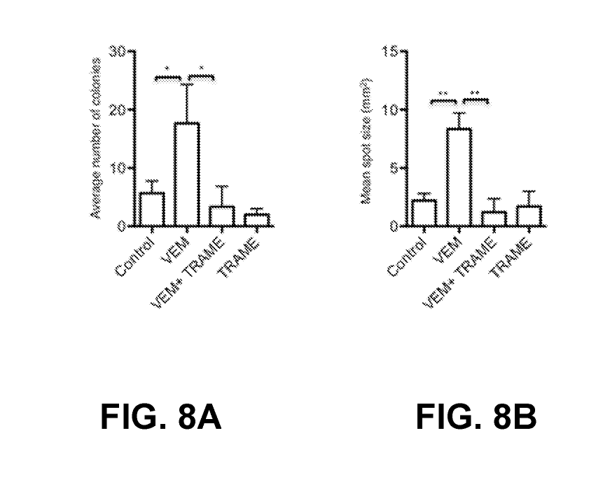

Furthermore, three-dimensional soft agar colony assays HEKa colonies proliferated upon exposure to vemurafenib, while the BRAF^{V600E} mutant melanoma line M249 had a decrease in colonies (FIG. 2D and FIG. 3B). HEKa colonies not only increased in number, but their mean spot sizes also increased significantly (p=0.007 by t-test, FIG. 2E). Addition of trametinib decreased the number and size of HEKa colonies induced by vemurafenib (FIGS. 2F, 8A-8B). Using these cultures, MAPK signaling was analyzed by western blot (FIGS. 2G-2H); and pERK and cell proliferation were analyzed by quantitative intracellular flow (phosphoflow) cytometry (FIG. 2I and FIGS. 4A-4B). Vemurafenib decreased pERK and cell cycle arrest in the BRAF^{V600E} mutant human melanoma cell line M249, while there was a paradoxical increase in pERK and cell cycle progression in HEKa cells (p=0.0225 by t-test). Furthermore, in the presence of vemurafinab the proliferative marker Ki67 decreased in M249 melanoma cells while it increased in HEKa cells (FIGS. 21-2J).

### Example 3: BRAF Inhibitor Enhances Healing in Skin Wounds Due to Paradoxical Proliferation of Epithelial Cells

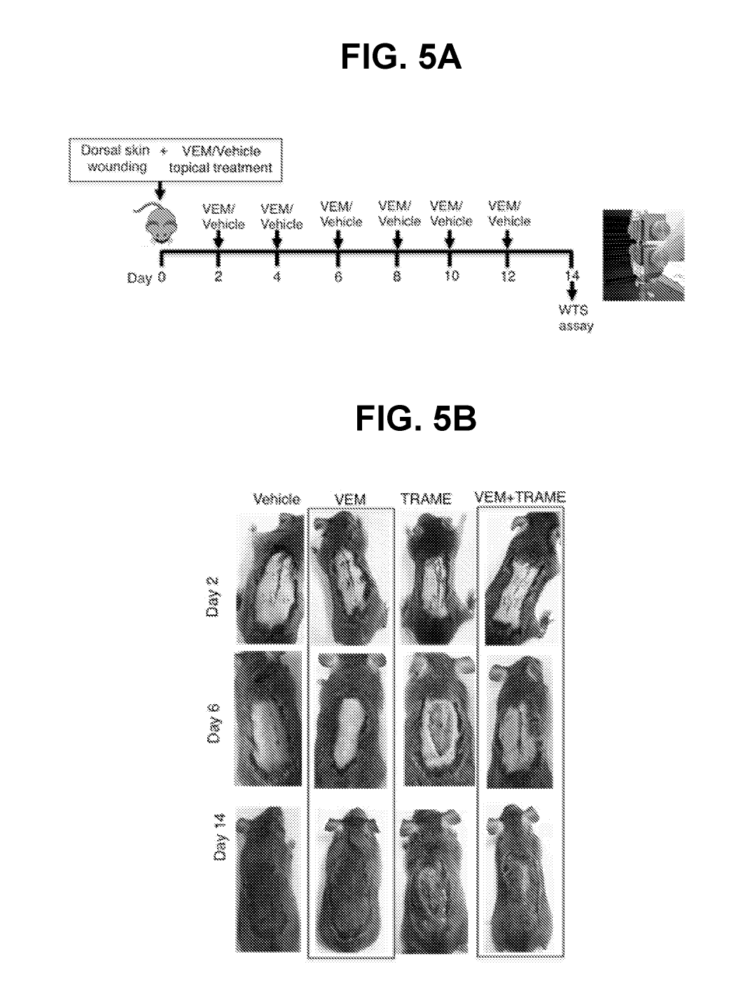

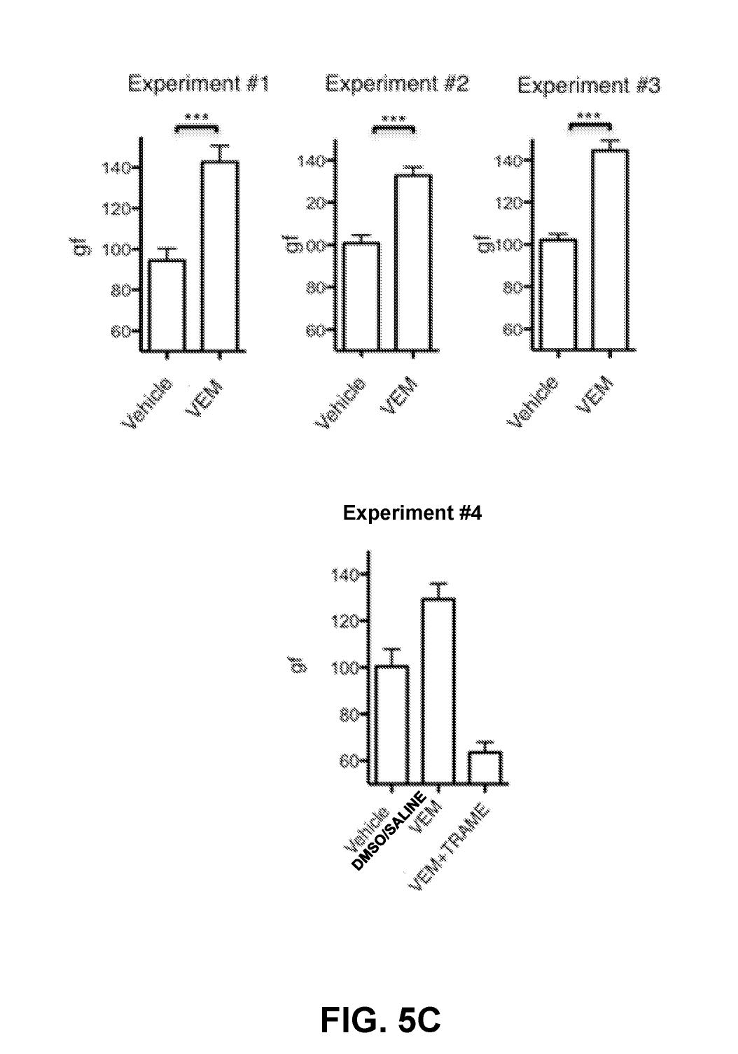

In a controlled wound-healing assay in an incisional wound healing mice model (FIG. 5A), a 2.5 cm dorsal skin wound was induced and was filled with either vehicle control (DMSO/saline) or a suspension of 2 mM of vemurafenib (obtained by crushing clinical grade pills of this agent) in vehicle. The skin wounds were surgically clipped on day 0 and mice were followed until day 14 (FIGS. 5A-5B). Over this time, the vemurafenib or vehicle control was applied topically every other day to 24 mice in the test group or to 24 mice in the control group, respectively, for a total of seven doses per mouse. On day 14, the mice were euthanized and the skin containing the wound was removed and mounted in 20 mm strips with a horizontal wound sample in each strip. The wound tensile strength (WTS, in gram force per 2 mm-gf/2 mm) was analyzed using a tensiometer that stretched the strips and recorded the WTS. In three independent replicate experiments (eight mice per group in each experiment; seven strips per wound), mice treated with vemurafenib had statistically significant improvements in the WTS compared to vehicle-treated controls (52%, 33% and 42%, respectively; p<0.0001 by t-test for all three experiments; FIG. 5C, Experiments #1-3). The administration of trametinib in addition to vemurafenib reduced the WTS by 51% compared to when using vemurafenib alone (p<0.0001 by t-test). In a separate cutaneous wound-healing assay, the 37% improvement in WTS by treatment with vemurafenib (p=0.01 by t-test vs. vehicle control) was partially reversed by the addition of 1 mg/kg of trametinib (FIG. 5B, “TRAME”, “VEM+TRAME”). For these wounds, the WTS decreased to 29% compared to vehicle control (p<0.0001 by t-test; FIG. 5C, Experiment #4).

The area of the wounds and their surroundings were analyzed histologically by two pathologists blinded to the study groups. Vemurafenib-treated wounds displayed accelerated proliferative stage of wound healing as evidenced by quantifying the extent of epidermal hyperplasia on both sides of the healing wounds. As shown in FIG. 3D, no re-epithelialization was observed in the tarmetinib-alone group (“TRAME”) or in the group treated with a combination of vermurafenib and trametinib (“VEM+TRAME”).

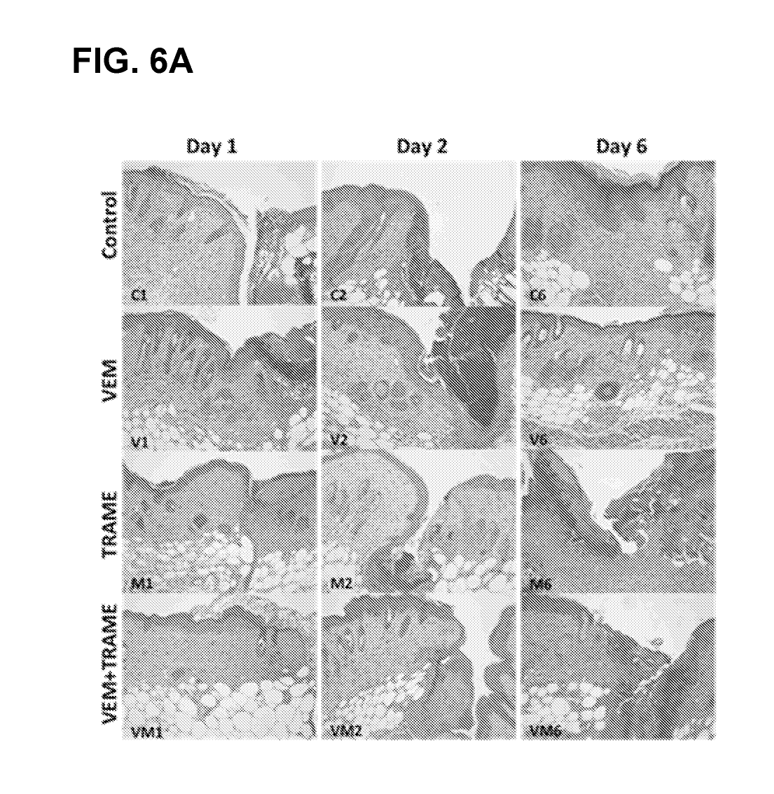

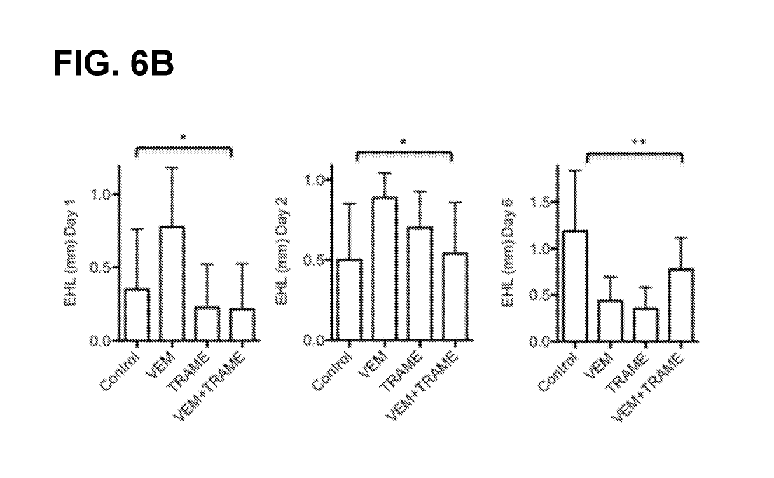

The area of the wounds and their surroundings were analyzed histologically by H&E staining by two pathologists and the extent of epidermal hyperplasia on both sides of the healing wounds was measured on days 1, 2 and 6 post-treatment (FIGS. 6A and 6B). On day 1 post-incision, wound-adjacent epidermal inflammation was more extensive in the presence of vemurafenib, with strong and rapid re-epithelialization starting at day 2. By day 6, surface integrity was re-established in the vemurafenib-treated group, whereas no evidence of dermal reparative fibrosis was observed in the mice treated with vehicle, trametinib or combination. No signs of healing or re-epithelialization were observed in the trametinib- or vemurafenib and trametinib-treated mice, and the wounds were ulcerated, specially, the ones treated with trametinib alone (FIG. 6A). On day 1 and 2, skin from the vemurafenib group tended to display epidermal hyperplasia over a greater distance than the other treated groups (p=0.0132 and p=0.0338 by one-way ANOVA, respectively), while by day 6 the vemurafenib group had less epidermal hyperplasia, consistent with a more rapid wound resolution (p=0.0012 by one way ANOVA; FIG. 4B). By day 6, 79% of control wounds showed re-epithelialization, whereas 100% of vemurafenib-treated wounds were completely re-epithelialized. No re-epithelialization was observed in the trametinib alone and vemurafenib and trametinib combination groups.

### Example 4: Vermurafenib Enhances Re-Epithelialization in Mice where Skin Contraction is Prevented

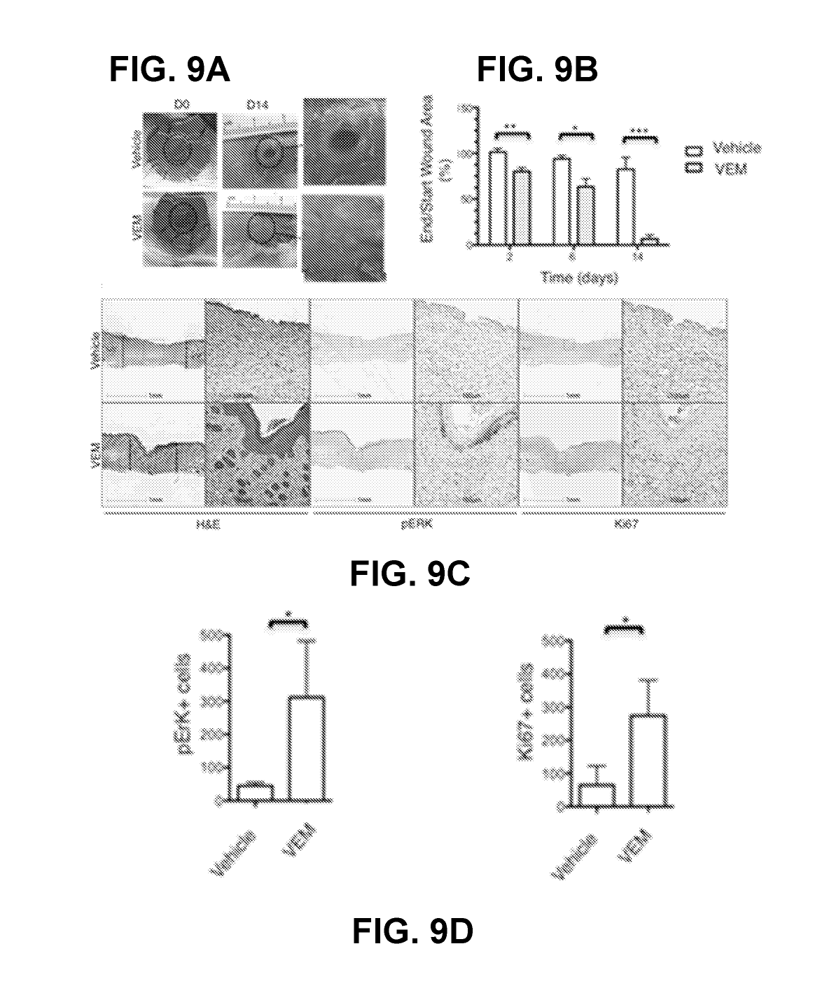

In an excisional wound splinting model in Balb/c mice (Wu, et al., Stem Cells 25, 2648-2659 (2007), 6-mm round wounds were induced on the back of mice; splinting rings were tightly adhered and sutured to the skin around the wounds, preventing wound closure caused by skin contraction. Vemurafenib (2 mg) or DMSO was applied on the wounds on days 0, 2 and 4, and percent wound closure was sequentially measured. As shown in FIGS. 9A-9B, the wounds treated with vemurafenib showed a significantly higher percentage of wound closure compared to the ones treated with vehicle on days 2, 6 and 14 (p=0.004, n=6; p=0.02, n=4; p=0.0002, n=6, respectively, by t-test). The area of the wounds and their surroundings were analyzed histologically and pERK+ and Ki67+ cells were quantified using digital pathology (FIGS. 9C-9D). Compared to controls, the healing process in the presence of vemurafenib was accelerated showing re-epithelialization by day 6 (FIGS. 10A-10B). The skin surface integrity was re-established by day 14 in the vemurafenib-treated wounds, whereas remodeling and dermal reparative fibrosis was delayed in the control group (FIG. 9C). The number of pERK+ and Ki67+ cells in the wounds treated with vemurafenib was statistically significantly higher by day 14 compared to the control group (p=0.02 by t-test; n=4; FIGS. 9C-9D), demonstrating paradoxical MAPK leading to enhanced epithelial cell proliferation.

### Example 5: Vemurafenib Upregulates MAPK and Wound Healing-Related Signatures

To further characterize epithelial skin repair, the skin samples obtained from the incisional wound healing model were analyzed by RNASeq. The list of differentially expressed genes on day 2, 6 and 14 is shown in Appendix 1, which is filed herewith. Appendix 1 includes a List of up-regulated and down-regulated genes in the wounds treated with vemurafenib at day 2, 6 and 14. The values listed are log₂ transformed after adding a pseudo FPKM value of 0.1 to remove large fold changes caused by low FPKM values (<0.1). The list of differentially expressed genes shown in Appendix 1 was compared to published data for the transcripts that were differentially modulated by blocking oncogenic MAPK signaling downstream of mutated BRAF^{V600E} using BRAF inhibitors (Nazarian et al. 2010), and to genes within the gene ontology term “wound healing” (GO:0042060) (Ashburner et al. 2000).

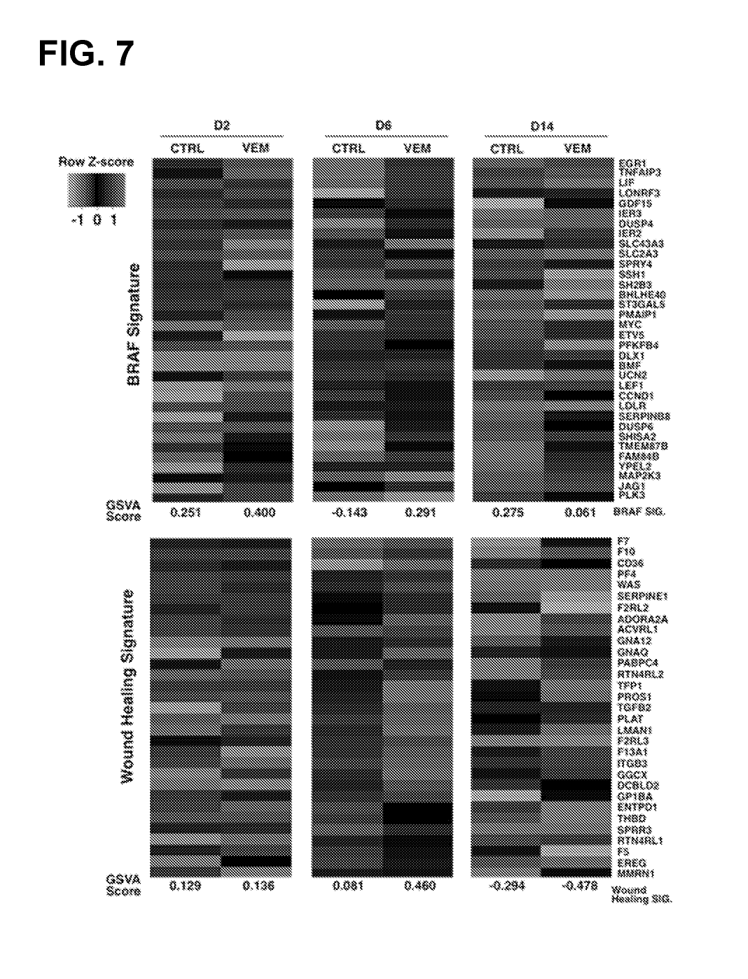

As shown by the gene expression heatmaps in FIG. 7, by day 2 (“D2”) there was a slight increase in the BRAF signature upon vemurafenib treatment but almost no change in the wound-healing signature. By day 6 (“D6”), both signatures were enriched significantly in the vemurafenib-treated samples compared to their respective controls. A more pronounced decrease on both the BRAF and wound-healing signatures was observed in the vemurafenib-treated wounds by day 14, consistent with a more rapid healing. The Gene Set Variation Analysis (GSVA) enrichment scores of the signatures showed the same trend (overall enrichment scores were computed based on Single-sample Gene Set Variation Analysis (GSVA)-Hanzelmann et al. 2013).

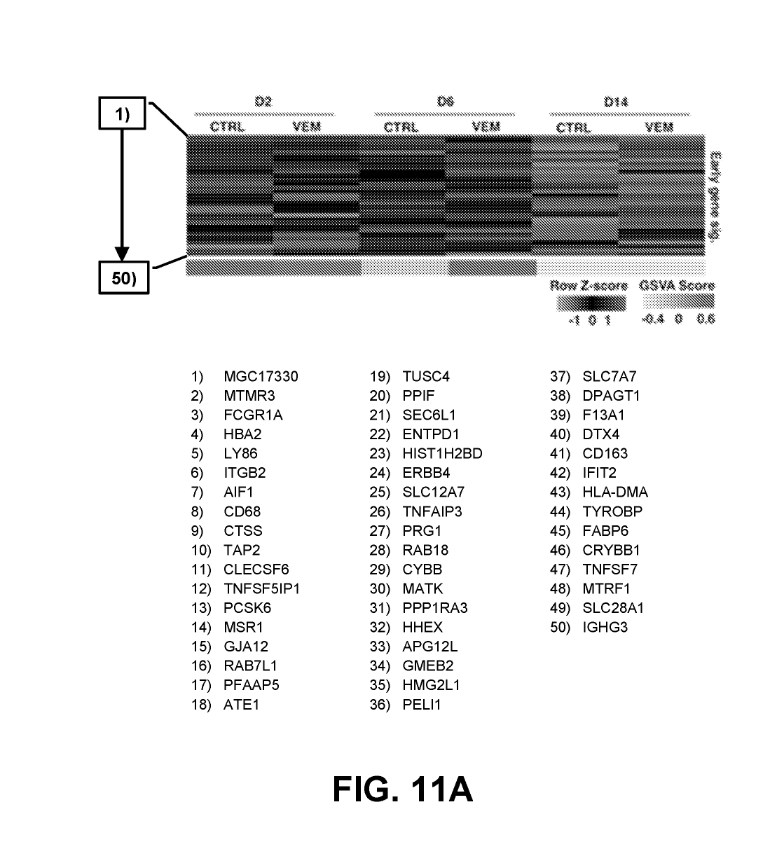

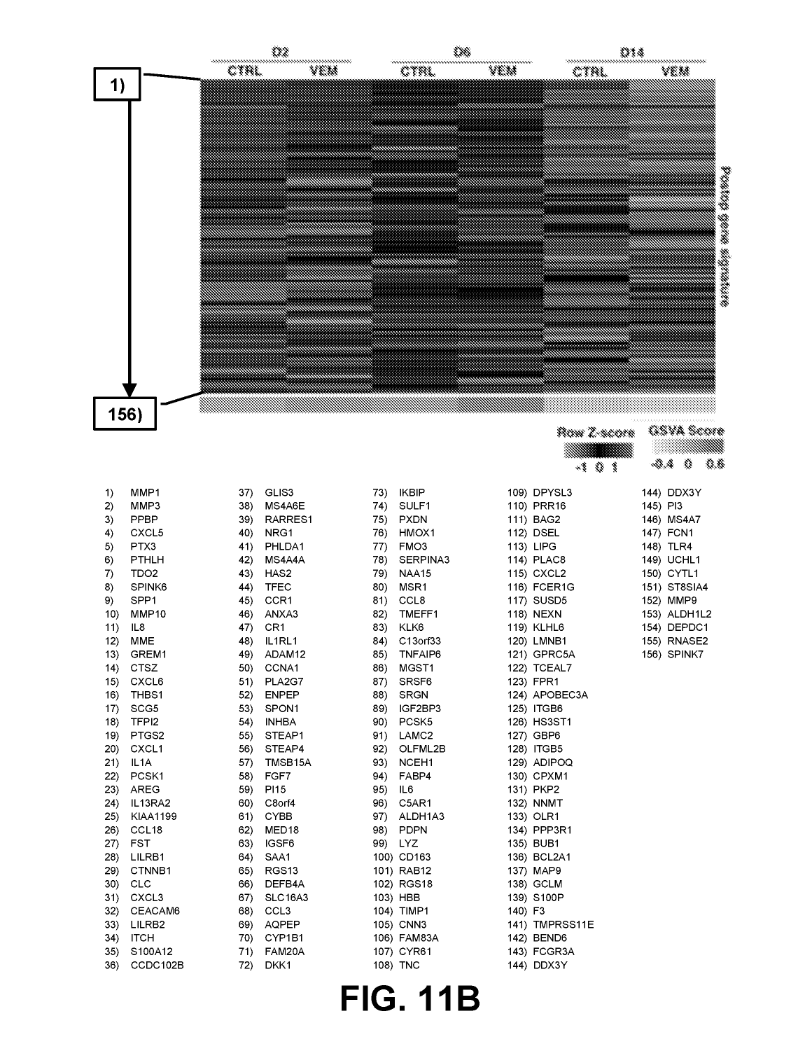

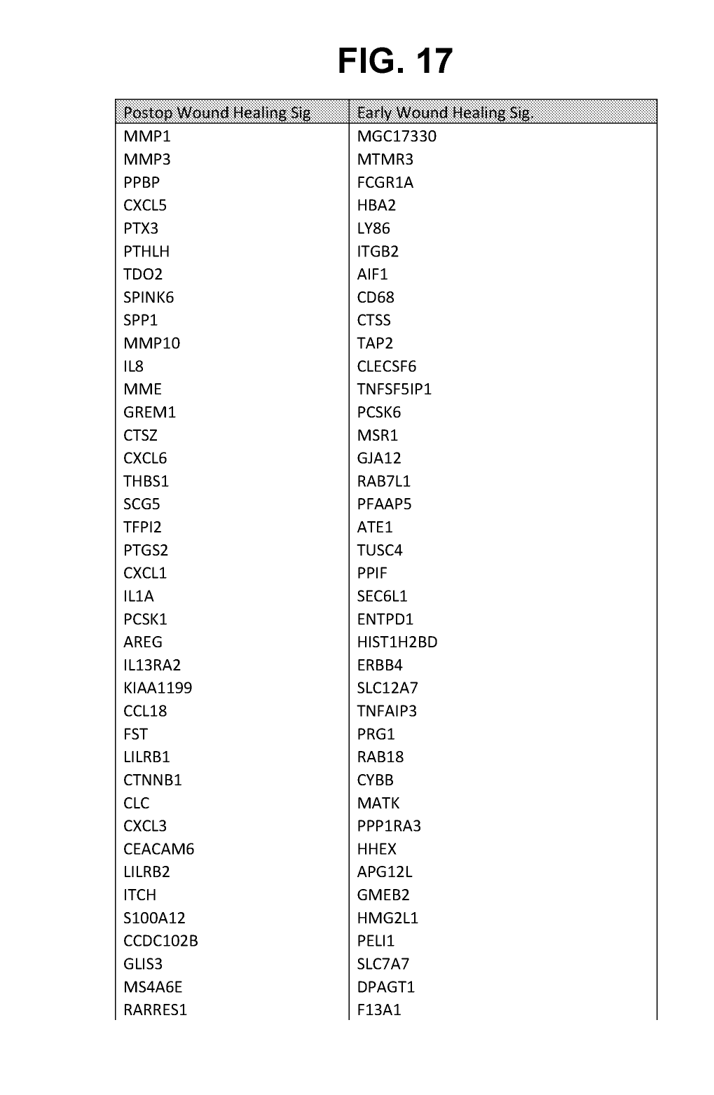

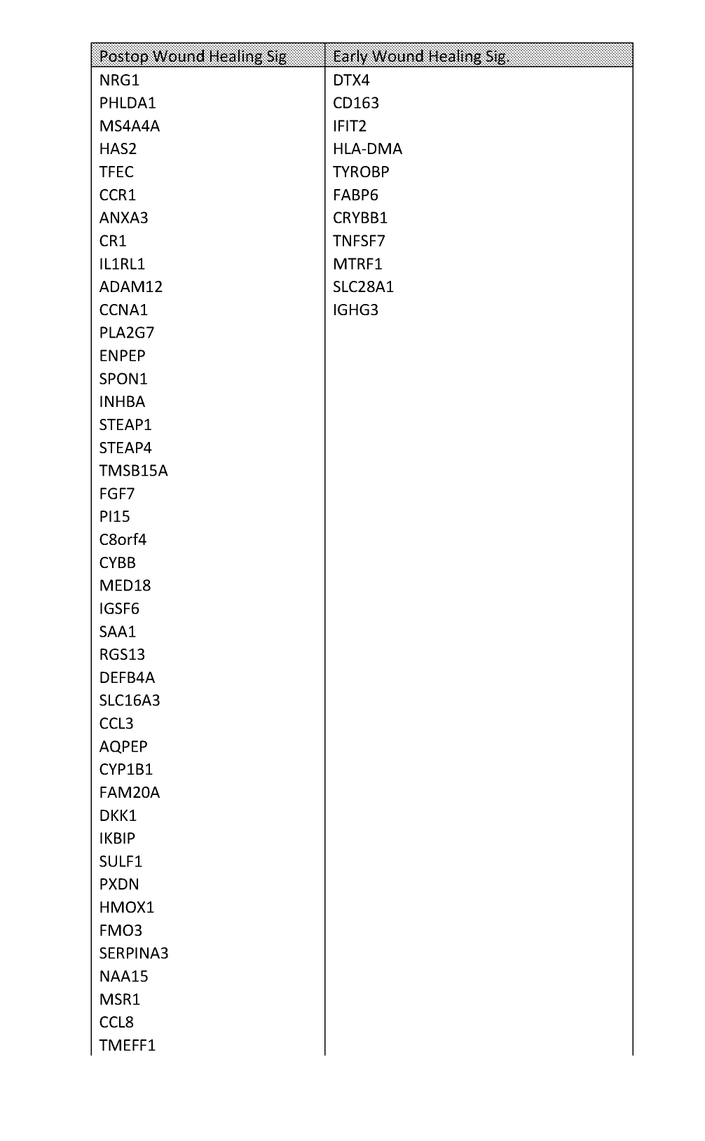

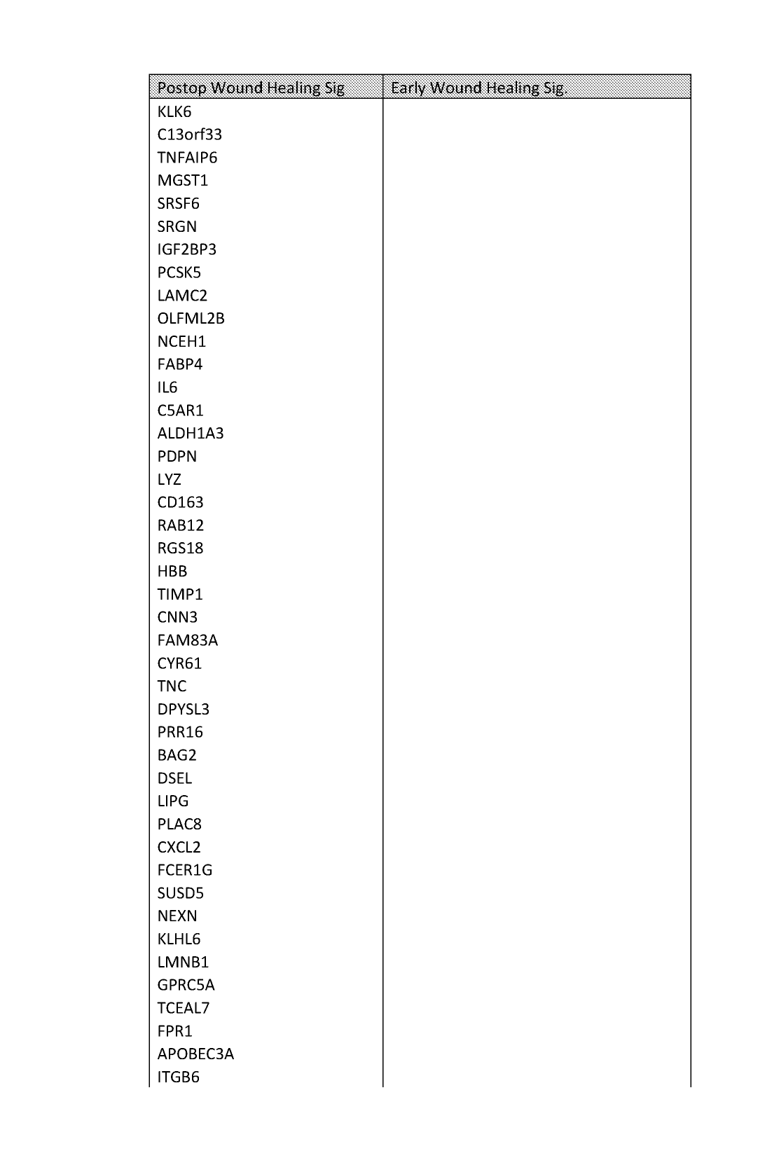

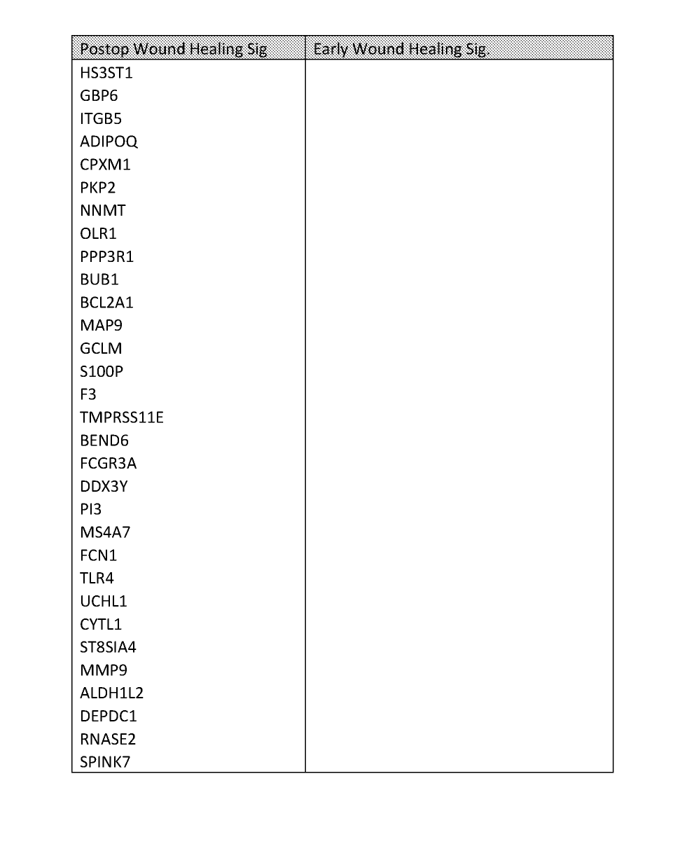

Additionally, the gene output was compared to an early stage wound healing signature, with mostly pro-inflammatory genes involved in the first stages of wound healing (Deonarine, K. et al., J. Transl. Med. 5:11 (2007)), and a post-operatory wound healing signature (Inkeles, M. S. et al., J. Invest. Dermatol. 135:151-159 (2015)). As shown in FIGS. 11A-11B and FIG. 17, these two wound-healing signatures were also enriched by day 6 in the vemurafenib-treated groups as opposed to the control group.

Genes recognized to be associated with wound healing (Fitsialos, G. et al., J. Biol. Chem. 282:15090-102 (2007)), were upregulated in vemurafenib-accelerated wound healing wounds compared to control wounds (FIGS. 7, 11A-11B).

### Example 6: Transcriptional Signatures Specific to Leukocytes, Endothelial Cells and Fibroblasts are Enriched in Vemurafenib-Treated Wounds

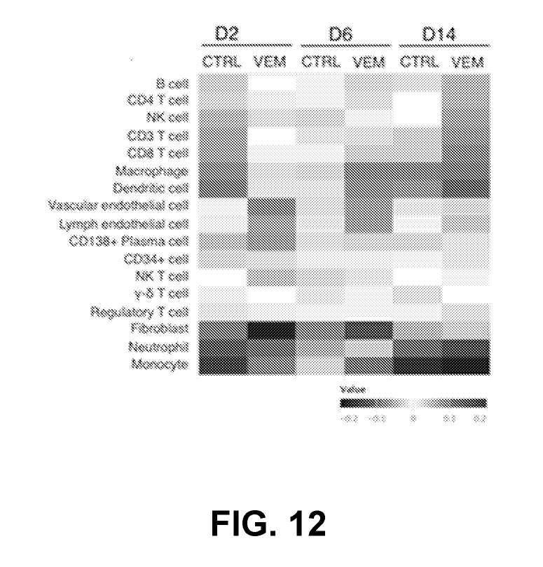

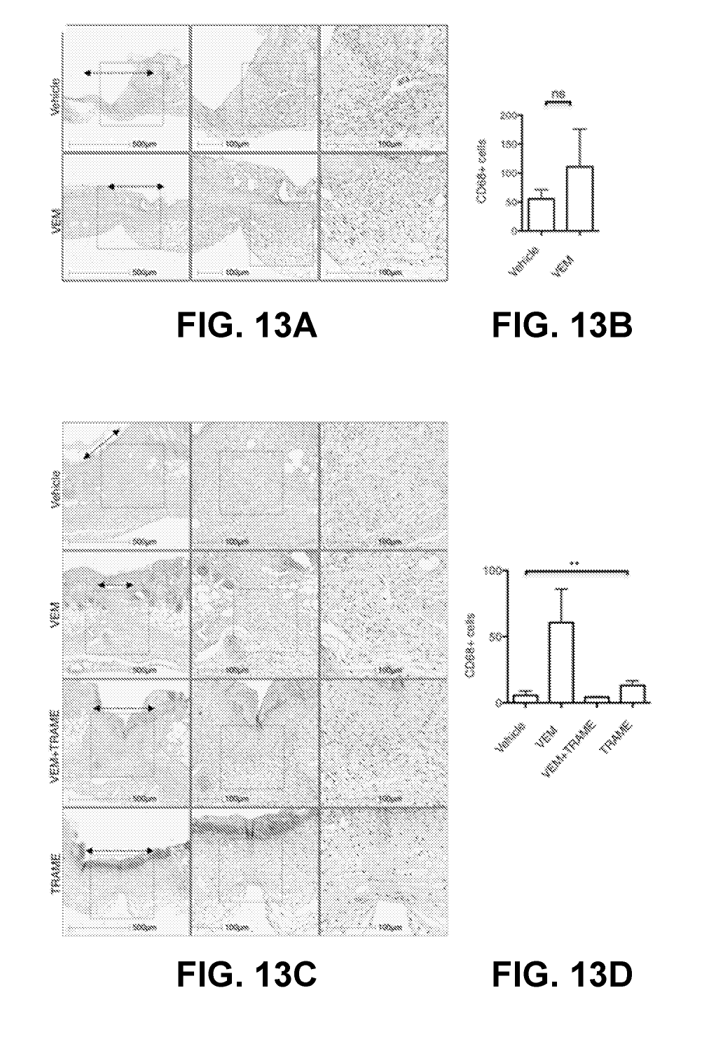

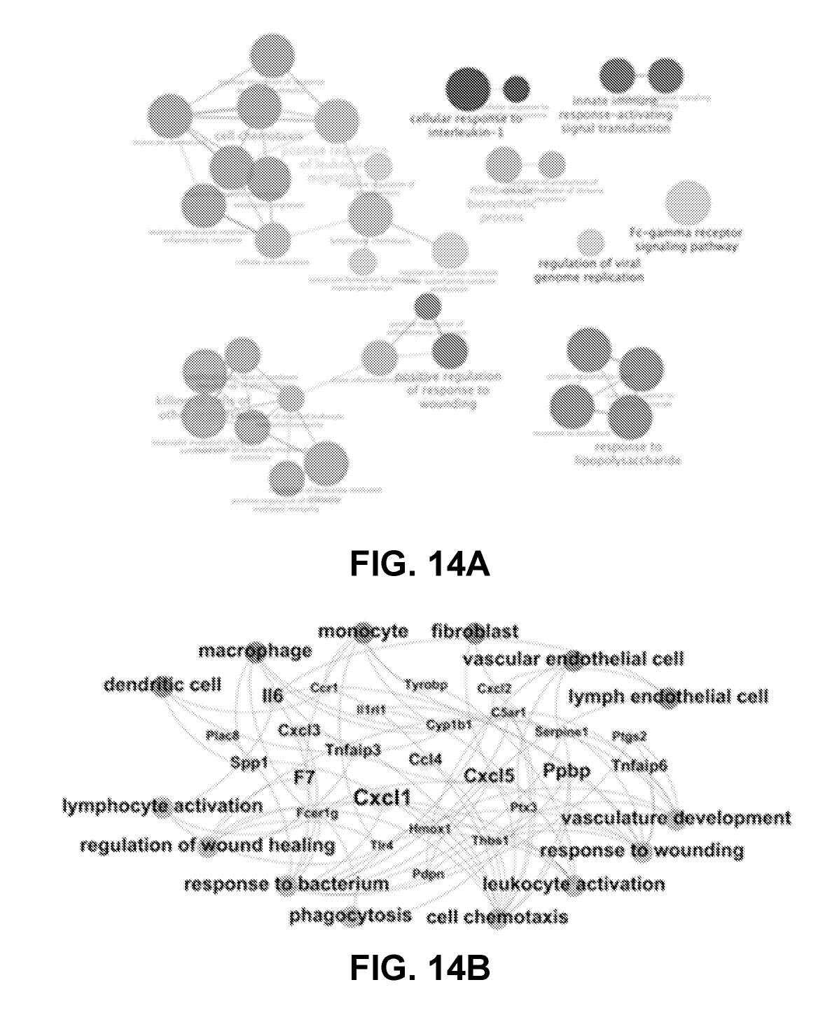

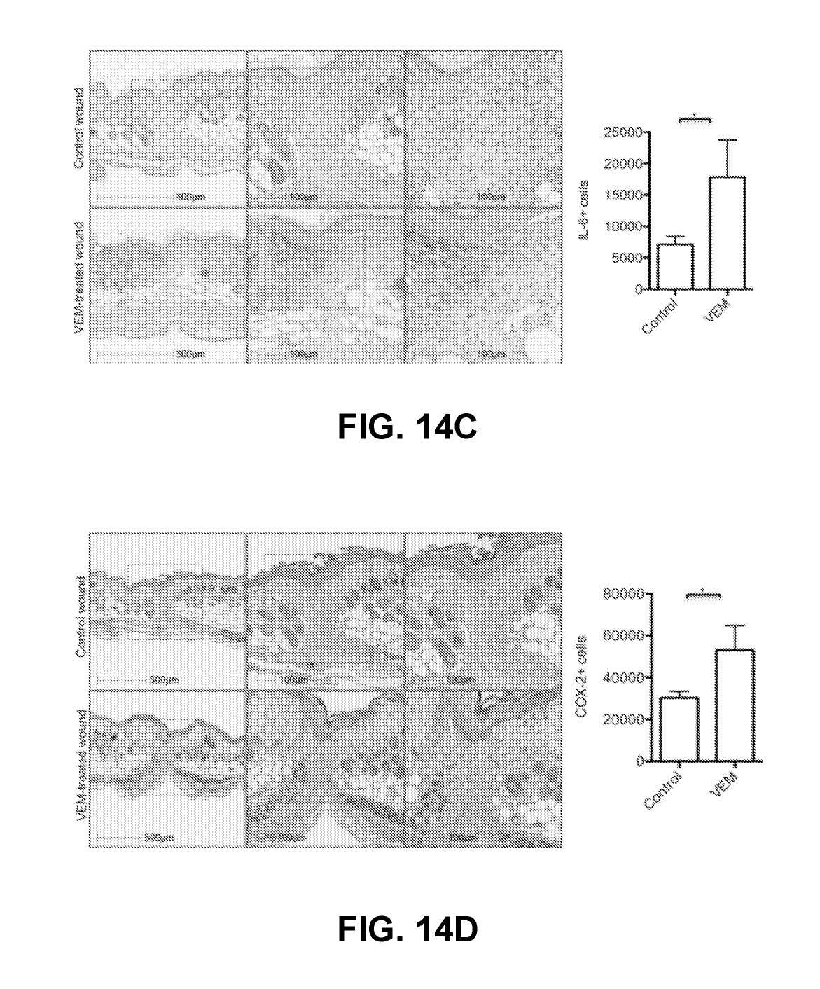

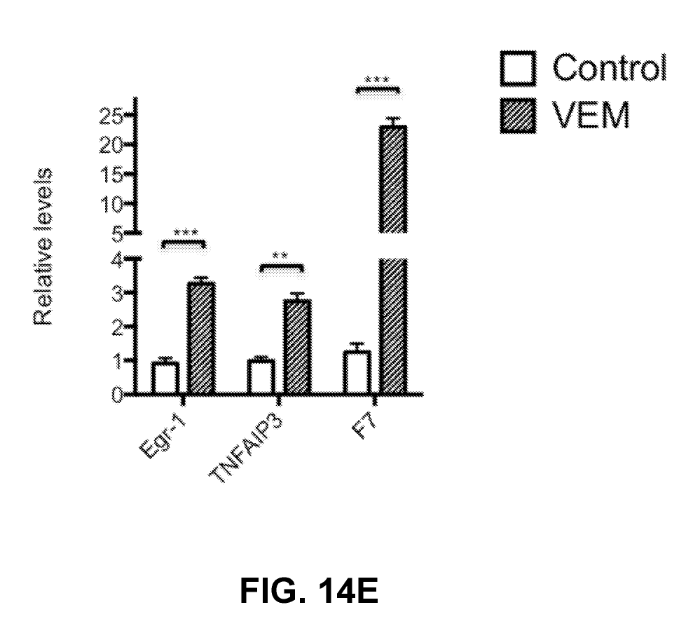

Enrichment of signatures for dendritic cells, macrophages, monocytes, fibroblasts, and vascular and lymphatic endothelial cells was observed in the wounds treated with vemurafenib as compared to the control wounds (FIG. 12). The increase in macrophages with topical vemurafenib was confirmed by IHC analysis (FIGS. 13A-13D). The increase in macrophages was abolished with the co-administration of trametinib. To elaborate on the activated pathways associated with the increased presence of wound healing cell subsets at this time point, the genes up-expressed in the vemurafenib-treated wounds were analyzed for enriched GO biological processes 2 fold increase) using ClueGo (Bindea, G. et al., Bioinformatics 25:1091-93 (2009)). Pathways involved in lymphocyte activation, vascular development and response to wounding were clearly found (FIGS. 14A-14B). Integration of the enrichments of cell subset signatures, gene and pathway upregulation showed enhanced recruitment of wound healing-specific cell subsets which results in stronger activation of the inflammatory and angiogenic wound healing processes (FIG. 14B). To confirm these findings, the levels of COX-2+ and IL-6+ cells were quantified by immunohistochemistry. The wounds treated with vemurafenib had higher IL-6+ and COX-2+ cells at the proliferation stage, on day 6 (FIGS. 14C-14D), which is consistent with the integrated RNAseq data shown in FIG. 14B. As a further verification of the RNAseq data, RTPCR for Egr-1, TNFAIP3 and F7, for total skin wounds, was performed. These three genes are upregulated in the gene signature (FIG. 5b) and the results on the RT-PCR verified this increase in vemurafenib-treated wounds by day 6 post-treatment (FIG. 14E).

### Example 7: Angiogenesis is Enhanced in Wounds Treated with Vemurafenib and Inhibited when Adding Trametinib

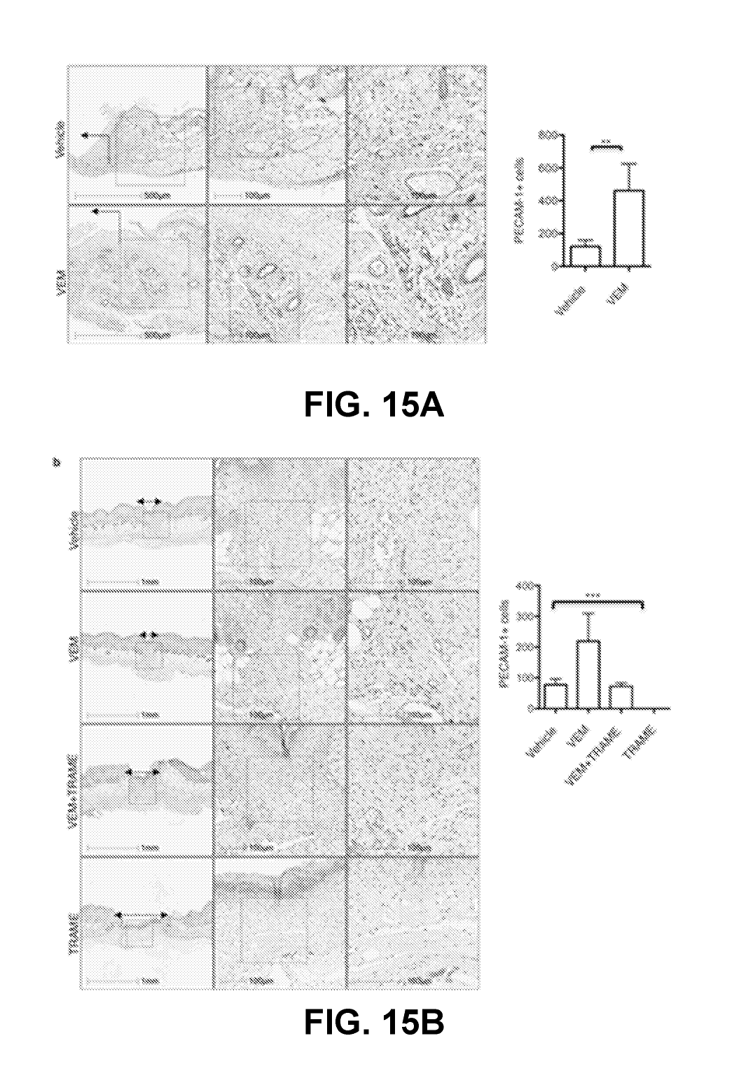

To confirm the beneficial effects of vemurafenib treatment on angiogenesis, PECAM-1+ cells in the excisional and incisional wound areas were quantified. In the excisional wound model there was a 74% increase in the number of PECAM-1+ cells in vemurafenib-treated wounds compared to the control wounds at day 6 post-treatment (p=0.006 by t-test n=4; FIG. 15A). A 65% increase of PECAM-1+ cells was observed in the incisional wound model in wounds treated with vemurafenib (p=0.02 by t-test), consistent with the enrichment of endothelial cells observed in the RNASeq data (FIG. 15B). This increase was reversed by the addition of topical trametinib (0.2 mg), with a 67% decrease in the number of PECAM-1+ cells at day 6 (p=0.01 by t-test; vemurafenib vs vemurafenib+trametinib). Trametinib alone completely depleted PECAM-1+ cells (FIG. 15B).

### Example 8: Vemurafenib does not Induce Skin Epidermal Tumors when Applied Topically in Mice

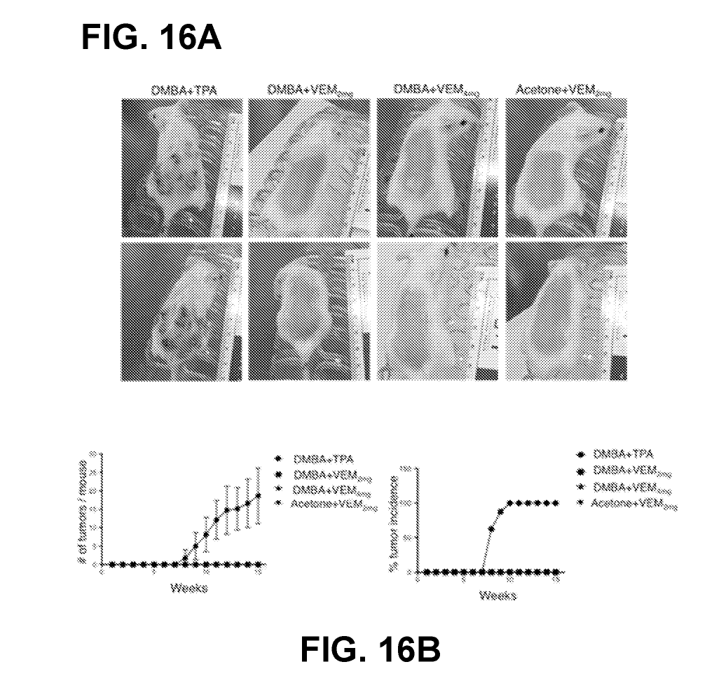

In order to analyze the possibility of a cutaneous tumor-promoting activity of topically applied vemurafenib, the well-established two-stage skin carcinogenesis model (e.g., Balmain, et al., Nature 307:658-60 (1984); Escuin-Ordinas, et al., Mol. Oncol. 8:250-60 (2014)) was used. Briefly, topical application of DMBA or acetone was followed either with topical TPA as a positive control for skin carcinogenesis or with topical vemurafenib, twice a week for 15 weeks. The only mice that developed skin carcinogenesis were the group treated with DMBA plus TPA (FIG. 16A; “DMBA+TPA”). No skin papillomas, keratoacanthomas or squamous cell carcinomas occurred in mice treated with DMBA plus vemurafenib at the concentrations that enhanced wound healing (2 mg) or higher (4 mg) (FIG. 16A, middle two columns). Therefore, topical vemurafenib did not promote skin carcinogenesis even after mice were exposed to an initiating carcinogen application that results in the presence of epithelial cells bearing RAS mutations (FIG. 16B).

Collectively, the studies described in the working Examples above demonstrate that the phenomenon of paradoxical MAPK activation by BRAF inhibitors may be exploited to enhance skin wound healing. Topical application of a BRAF inhibitor resulted in the accelerated healing of skin wounds by primarily acting on the proliferative stage of wound healing, with improvement in multiple other events in wound healing including inflammation and angiogenesis. These benefits were offset by adding a MEK inhibitor that is known to inhibit paradoxical MAPK activation, while this topical therapy did not result in tumor promotion. Therefore, topical BRAF inhibitor could be used to accelerate the healing of acute skin wounds, such as abrasions and surgical incisions.

### REFERENCES

The references, patents and published patent applications listed below, and all references cited in the specification above are hereby incorporated by reference in their entirety, as if fully set forth herein.
V. R. Belum, A. Fischer, J. N. Choi, M. E. Lacouture, Dermatological adverse events from BRAF inhibitors: a growing problem. Current oncology reports 15, 249 (June, 2013).
B. Comin-Anduix et al., Modulation of cell signaling networks after CTLA4 blockade in patients with metastatic melanoma. PLoS One 5, e12711 (2010).
H. Escuin-Ordinas et al., COX-2 inhibition prevents the appearance of cutaneous squamous cell carcinomas accelerated by BRAF inhibitors. Mol Oncol, (March 2014).
K. T. Flaherty et al., Combined BRAF and MEK inhibition in melanoma with BRAF V600 mutations. The New England journal of medicine 367, 1694 (November, 2012).
R. Gorodetsky, W. H. McBride, H. R. Withers, Assay of radiation effects in mouse skin as expressed in wound healing. Radiation research 116, 135 (1988).
R. Gorodetsky, The use of fibrin based matrices and fibrin microbeads (FMB) for cell based tissue regeneration. Expert Opin Biol Ther 8, 1831 (December, 2008).
C. A. Hall-Jackson et al., Paradoxical activation of Raf by a novel Raf inhibitor. Chem Biol 6, 559 (August, 1999).
S. Hanzelmann, R. Castelo, J. Guinney, GSVA: gene set variation analysis for microarray and RNA-seq data. BMC Bioinformatics 14, 7 (2013).
G. Hatzivassiliou et al., RAF inhibitors prime wild-type RAF to activate the MAPK pathway and enhance growth. Nature 464, 431 (Mar. 18, 2010).
S. J. Heidorn et al., Kinase-dead BRAF and oncogenic RAS cooperate to drive tumor progression through CRAF. Cell 140, 209 (Jan. 22, 2010).
M. Holderfield et al., RAF inhibitors activate the MAPK pathway by relieving inhibitory autophosphorylation. Cancer Cell 23, 594 (May 13, 2013).
E. W. Joseph et al., The RAF inhibitor PLX4032 inhibits ERK signaling and tumor cell proliferation in a V600E BRAF-selective manner. Proceedings of the National Academy of Sciences of the United States of America 107, 14903 (Aug. 17, 2010).
D. Kim et al., TopHat2: accurate alignment of transcriptomes in the presence of insertions, deletions and gene fusions. Genome Biol 14, R36 (2013).
J. Larkin et al., Combined Vemurafenib and Cobimetinib in BRAF-Mutated Melanoma. New England Journal of Medicine (in press), (2014).
G. V. Long et al., Combined BRAF and MEK Inhibition Versus BRAF Inhibition Alone in Melanoma. New England Journal of Medicine (in press), (2014).
R. Nazarian et al., Melanomas acquire resistance to B-RAF(V600E) inhibition by RTK or N-RAS upregulation. Nature 468, 973 (Dec. 16, 2010).
P. A. Oberholzer et al., RAS Mutations Are Associated With the Development of Cutaneous Squamous Cell Tumors in Patients Treated With RAF Inhibitors. Journal of clinical oncology: official journal of the American Society of Clinical Oncology 30, 316 (Jan. 20, 2012).
P. I. Poulikakos, C. Zhang, G. Bollag, K. M. Shokat, N. Rosen, RAF inhibitors transactivate RAF dimers and ERK signalling in cells with wild-type BRAF. Nature 464, 427 (Mar. 18, 2010).
F. Su et al., RAS mutations in cutaneous squamous-cell carcinomas in patients treated with BRAF inhibitors. The New England journal of medicine 366, 207 (Jan. 19, 2012).
J. Zavadil et al., Genetic programs of epithelial cell plasticity directed by transforming growth factor-beta. Proc Natl Acad Sci USA 98, 6686 (Jun. 5, 2001).
G. C. Gurtner et al., Wound repair and regeneration. Nature 453, 314-321 (2008).
B. K. Sun et al., Advances in skin grafting and treatment of cutaneous wounds. Science 346, 941-945 (2014).
Y. Wu et al., Mesenchymal stem cells enhance wound healing through differentiation and angiogenesis. Stem Cells 25, 2648-2659 (2007).
M. Ashburner et al., Gene ontology: tool for the unification of biology. The Gene Ontology Consortium. Nat Genet 25, 25-29 (2000).
K. Deonarine et al., Gene expression profiling of cutaneous wound healing. J Transl Med 5, 11 (2007).
M. S. Inkeles et al., Comparison of molecular signatures from multiple skin diseases identifies mechanisms of immunopathogenesis. J Invest Dermatol 135, 151-159 (2015).
G. Fitsialos et al., Transcriptional signature of epidermal keratinocytes subjected to in vitro scratch wounding reveals selective roles for ERK1/2, p38, and phosphatidylinositol 3-kinase signaling pathways. J Biol Chem 282, 15090-15102 (2007).
G. Bindea et al., ClueGO: a Cytoscape plug-in to decipher functionally grouped gene ontology and pathway annotation networks. Bioinformatics 25, 1091-1093 (2009).
A. Balmain et al., Activation of the mouse cellular Harvey-ras gene in chemically induced benign skin papillomas. Nature 307, 658-660 (1984).
W. R. Swindell et al., Dissecting the psoriasis transcriptome: inflammatory- and cytokine-driven gene expression in lesions from 163 patients. BMC Genomics 14, 527 (2013).
M. Jacomy et al., ForceAtlas2, a continuous graph layout algorithm for handy network visualization designed for the Gephi software. PLoS One 9, e98679 (2014).
E. L. Abel et al., Multi-stage chemical carcinogenesis in mouse skin: fundamentals and applications. Nature protocols 4, 1350-1362 (2009).
T. O. Ishikawa et al., Cox-2 gene expression in chemically induced skin papillomas cannot predict subsequent tumor fate. Mol Oncol 4, 347-356 (2010).

1. A method of treating a wound caused by a disorder or condition comprising:
contacting the wound on a subject with an effective amount of a BRAF inhibitor, wherein the subject is suffering from the disorder or condition.

2. The method of claim 1, wherein the disorder or condition is epidermolysis bullosa (EB), Stevens-Johnson Syndrome (SJS), Toxic Epidermal Necrolysis (TEN), staphylococcal scaled skin syndrome (SSSS), Pemphigus vulgaris (PV), or toxic shock syndrome (TSS).

3. The method of claim 1, wherein the BRAF inhibitor has a structure according to any one of Formulas (I)-(IV) or a pharmaceutically acceptable salt thereof.

4. The method of claim 1, wherein the BRAF inhibitor is selected from the group consisting of AMG542, ARQ197, ARQ736, AZ628, CEP-32496, GDC-0879, GSK1120212, GSK2118436 (dabrafenib, Tafinlar®), LGX818 (encorafenib), NMS-P186, NMS-P349, NMS-P383, NMS-P396, NMS-P730, PLX3603 (R05212054), PLX4032 (vemurafenib, Zelboraf®), PLX4720 (Difluorophenyl-sulfonamine), PF-04880594, PLX4734, RAF265 (CHIR-265), R04987655, SB590885, sorafenib, sorafenib tosylate, and XL281 (BMS-908662).

5. The method of claim 1, wherein the BRAF inhibitor is part of a pharmaceutical composition, the pharmaceutical composition comprising:
an effective amount of the BRAF inhibitor; and
a pharmaceutically acceptable carrier.

6. The method of claim 5, wherein the pharmaceutical composition coats or impregnates a wound dressing.

7. The method of claim 6, wherein the wound dressing is a an alginate dressing, an antimicrobial dressing, a bandage, a Band-Aid®, a biosynthetic dressing, a biological dressing, a collagen dressing, a composite dressing, a compression dressing, a contact layer dressing, a foam dressing, a gauze dressing, a hydrocolloid dressing, a hydrogel dressing, a skin sealant or liquid skin dressing, a specialty absorptive dressing, a transparent film dressing, or a wound filler.

8. The method of claim 1, wherein contacting the wound is accomplished by topical administration of an ointment, cream liquid, gel, hydrogel, or a spray.

9. The method of claim 1, further comprising administering a second pro-angiogenic agent in combination with the BRAF inhibitor.

10. The method of claim 9, wherein the a second pro-angiogenic agent is a fibroblast growth factor, vascular endothelial growth factor (VEGF), platelet-derived growth factor (PDGF), placental growth factor (PIGF), an angiopoietin, a matrix metalloproteinase (MMP), delta-like ligand 4 (Dll4), or a class 3 Semaphorin (SEMA3).

11. The method of claim 1, wherein the BRAF inhibitor is LGX818 (encorafenib), GSK2118436 (dabrafenib, Tafinlar®), or PLX4032 (vemurafenib, Zelboraf®).

12.-13. (canceled)

14. The method of claim 1, wherein the BRAF inhibitor has increased MAPK activation activity.

15. A pharmaceutical composition for treating a wound comprising:
an effective amount of a BRAF inhibitor;
a second pro-angiogenic agent; and
a pharmaceutically acceptable carrier.

16. The pharmaceutical composition of claim 15, wherein the BRAF inhibitor has a structure according to any one of Formulas (I)-(IV) or a pharmaceutically acceptable salt thereof.

17. The pharmaceutical composition of claim 15, wherein the BRAF inhibitor is selected from the group consisting of AMG542, ARQ197, ARQ736, AZ628, CEP-32496, GDC-0879, GSK1120212, GSK2118436 (dabrafenib, Tafinlar®), LGX818 (encorafenib), NMS-P186, NMS-P349, NMS-P383, NMS-P396, NMS-P730, PLX3603 (R05212054), PLX4032 (vemurafenib, Zelboraf®), PLX4720 (Difluorophenyl-sulfonamine), PF-04880594, PLX4734, RAF265 (CHIR-265), R04987655, SB590885, sorafenib, sorafenib tosylate, and XL281 (BMS-908662).

18. The pharmaceutical composition of claim 15, wherein said pharmaceutical composition is a topical agent comprising an ointment, cream liquid, gel, hydrogel, or a spray.

19. The pharmaceutical composition of claim 15, wherein the second pro-angiogenic agent is a fibroblast growth factor, vascular endothelial growth factor (VEGF), platelet-derived growth factor (PDGF), placental growth factor (PIGF), an angiopoietin, a matrix metalloproteinase (MMP), delta-like ligand 4 (Dll4), or a class 3 Semaphorin (SEMA3).

20. The pharmaceutical composition of claim 15, wherein the pharmaceutical composition is impregnated in or coats a wound dressing.

21. The pharmaceutical composition of claim 20, wherein the wound dressing is an alginate dressing, an antimicrobial dressing, a bandage, a Band-Aid®, a biosynthetic dressing, a biological dressing, a collagen dressing, a composite dressing, a compression dressing, a contact layer dressing, a foam dressing, a gauze dressing, a hydrocolloid dressing, a hydrogel dressing, a skin sealant or liquid skin dressing, a specialty absorptive dressing, a transparent film dressing, or a wound filler.

22. The pharmaceutical composition of claim 15, wherein the BRAF inhibitor is LGX818 (encorafenib), GSK2118436 (dabrafenib, Tafinlar®), or PLX4032 (vemurafenib, Zelboraf®).

23.-25. (canceled)
